(19) United States
(12) Patent Application Publication
(10) Pub. No.: US 2021/0187130 A1
(43) Pub. Date: Jun. 24, 2021

(54) PET RADIOPHARMACEUTICALS FOR NON-INVASIVE EVALUATION OF HIF-2alpha

(71) Applicant: THE BOARD OF REGENTS OF THE UNIVERSITY OF TEXAS SYSTEM, Austin, TX (US)

(72) Inventors: James BRUGAROLAS, Irving, TX (US); Xiankai SUN, Coppell, TX (US); Sashi DEBNATH, Dallas, TX (US)

(73) Assignee: THE BOARD OF REGENTS OF THE UNIVERSITY OF TEXAS SYSTEM, Austin, TX (US)

(21) Appl. No.: 17/153,128

(22) Filed: Jan. 20, 2021

Related U.S. Application Data
(63) Continuation-in-part of application No. 17/129,352, filed on Dec. 21, 2020.
(60) Provisional application No. 62/950,556, filed on Dec. 19, 2019.
(60) Provisional application No. 62/967,522, filed on Jan. 29, 2020.
(60) Provisional application No. 62/981,195, filed on Feb. 25, 2020.

(51) Int. Cl. A61K 51/04; A61K 49/00; A61B 6/03; A61B 6/00

(52) U.S. Cl. CPC A61K 51/04; A61B 6/507; A61B 6/037; A61K 49/0004

(57) ABSTRACT

Provided herein are hypoxia inducible factor 2-alpha (HIF-2α)-specific radioactive tracers, methods of use thereof, and methods of synthesis thereof. Specifically, provided herein are HIF-2α-specific radioactive tracers comprising an HIF-2α-specific agent developed as a therapeutic inhibitor and a positron emitting radioactive label. Embodiments provide methods of detecting an HIF-2α-expressing tumor, detecting an HIF-2α inhibitor resistant tumor, evaluating a change in HIF-2α expression in response to an anti-cancer treatment, detecting acquisition of HIF-2α inhibitor resistance, evaluating efficacy of an HIF-2α depletion therapy, detecting or monitoring an ischemic area in a subject, or detecting and monitoring pulmonary hypertension in a subject. Also provided are methods of synthesizing an HIF-2α-specific radioactive tracer.

### PRIORITY

This application is a continuation-in-part of U.S. Ser. No. 17/129,352, filed December 21, 2020, and claims the benefit of U.S. Ser. No. 62/950,556, filed December 19, 2019, U.S. Ser. No. 62/967,522, filed January 29, 2020, U.S. Ser. No. 62/981,195, filed February 25, 2020, all of which are incorporated herein by reference in their entireties.

### BACKGROUND

Methods are needed in the art for imaging and monitoring of hypoxia inducible factor 2-alpha (HIF-2α) in cancerous tissues, ischemic tissues, lung tissue, and other tissues. Methods of querying HIF-2α expression across sites of disease and also dynamically over time and following different interventions (radiation or other systemic therapies) would be extremely valuable. In addition, probes are needed to monitor the acquisition of resistance in cancerous tissues, and in particular of gatekeeper mutations.

### SUMMARY OF THE DISCLOSURE

Provided herein are HIF-2α-specific radioactive tracers, methods of use thereof, and methods of synthesis thereof.

An embodiment provides a hypoxia inducible factor 2-alpha (HIF-2α)-specific radioactive tracer comprising an HIF-2α-specific inhibitor and a radioactive label, wherein the radioactive label is a positron emitting radioisotope. The positron emitting radioisotope can be ¹¹C or ¹⁸F. The HIF-2α-specific inhibitor can be PT2385, PT2399, PT2977 or other related structures binding the same pocket. The HIF-2α-specific radioactive tracer can be

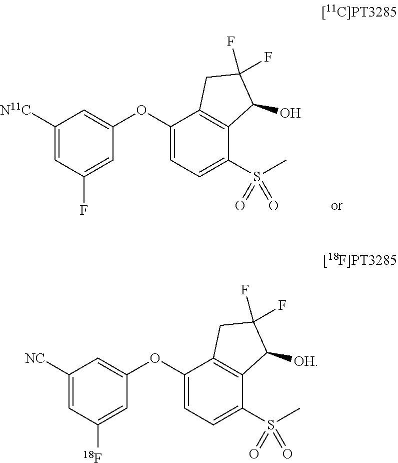

Another embodiment provides a method of detecting an HIF-2α-expressing tumor in a subject comprising administering to a subject having a tumor an HIF-2α-specific radioactive tracer, wherein the radioactive label is a positron emitting radioisotope; subjecting the subject to a positron emission topography (PET) scan; and determining an amount of the HIF-2α-specific radioactive tracer, wherein an increased amount of the tracer as compared to a control indicates an HIF-2α-expressing tumor. The HIF-2α-specific radioactive tracer can comprise an HIF-2α-specific inhibitor and a radioactive label.

An additional embodiment provides a method of detecting an HIF-2α inhibitor resistant tumor in a subject comprising administering to a subject having a tumor an HIF-2α-specific radioactive tracer comprising an HIF-2α-specific inhibitor and a radioactive label, wherein the radioactive label is a positron emitting radioisotope; subjecting the subject to a PET scan; and determining the amount of the HI F-2α-specific radioactive tracer, wherein a decreased amount of tracer as compared to a control indicates an HIF-2α inhibitor resistant tumor. The HIF-2α-specific radioactive tracer can comprise an HIF-2α-specific inhibitor and a radioactive label.

Another embodiment provides a method of evaluating a change in HIF-2α expression in a subject in response to an anti-cancer treatment comprising administering to a subject having a tumor an HIF-2α-specific radioactive tracer, subjecting the subject to a first PET scan, and determining a first amount of HIF-2α expression in the subject; administering to the subject an anti-cancer treatment; administering to the subject the HIF-2α-specific radioactive tracer; subjecting the subject to a second PET scan, and determining a second amount of HIF-2α expression in the subject; and comparing the first amount and second amount of HIF-2α expression. The anti-cancer treatment can comprise radiotherapy, chemotherapy, targeted therapy, immunotherapy, or a combination thereof. The targeted therapy can be an HIF-2α inhibitor. The steps of administering the HIF-2α-specific radioactive tracer, subjecting the subject to a second PET scan, and determining a second amount of HIF-2α expression in the subject; and comparing the first amount and second amount of HIF-2α expression can be repeated to determine changes in HIF-2α expression over time. A decreased second amount as compared to the first amount may indicate a decreased sensitivity of the tumor to an HIF-2α inhibitor therapy. The HIF-2α-specific radioactive tracer can comprise an HIF-2α-specific inhibitor and a radioactive label. The targeted therapy can be the administration of PT2385, PT2399, PT2977, a related compound, other related structures binding the same pocket, or a different class of compounds targeting HIF-2α.

Yet another embodiment provides a method of detecting acquisition of HIF-2α inhibitor resistance in a subject. The method comprises administering to the subject an HIF-2α-specific radioactive tracer and subjecting the subject to a first PET scan. A first baseline level of the HIF-2α-specific radioactive tracer is determined. The subject is administered an HIF-2α inhibitor. The subject is then administered an HIF-2α-specific radioactive tracer and subjected to a second PET scan. A second level of the HIF-2α-specific radioactive tracer is determined. The second level is compared to the first baseline level and where a second level of the HIF-2α-specific radioactive tracer is decreased as compared to the first baseline level, then there may be an acquisition of HIF-2α inhibitor resistance.

The acquisition of HIF-2α inhibitor resistance can be the acquisition of a somatic HIF-2α mutation.

In various embodiments, the tumor can be a clear cell renal cell carcinoma (ccRCC). The tumor can be a primary tumor or a metastasis. In an embodiment any tumor type or cancerous tissue type can be probed according to the methods described herein.

An embodiment provides a method of evaluating efficacy of an HIF-2α depletion therapy in a subject. The method comprises administering an HIF-2α-specific radioactive tracer to the subject and subjecting the subject to a first PET scan. A first baseline level of the HIF-2α-specific radioactive tracer is determined. The subject is administered an HIF-2α depletion therapy. The subject is then administered an HIF-2α-specific radioactive tracer and subjected to a second PET scan. A second level of the HIF-2α-specific radioactive tracer can be determined. The second level is compared to the first baseline level and where a second level of the HIF-2α-specific radioactive tracer is decreased as compared to the first baseline level, then there is efficacy of an HIF-2α depletion therapy.

The HIF-2α depletion therapy can comprise an siRNA targeting HIF-2α. Where a second level of the HIF-2α-specific radioactive tracer is equivalent or increased as compared to the first baseline level, then there is an acquisition of resistance to the HIF-2α depletion therapy, characterized by a restoration of HIF-2α levels.

An embodiment provides a method of detecting an ischemic area in a subject comprising administering to the subject an HIF-2α-specific radioactive tracer, wherein the radioactive label is a positron emitting radioisotope; subjecting the subject to a PET scan; and determining the amount of the tracer, wherein an increased amount of the tracer indicates an ischemic area. The method can further comprise administering to the subject having an ischemic area an anti-ischemia treatment. The HIF-2α-specific radioactive tracer can comprise an HIF-2α-specific inhibitor and a radioactive label.

Another embodiment provides a method of monitoring an ischemic area in a subject comprising administering to a subject having an ischemic area an HIF-2α-specific radioactive tracer; subjecting the subject to a first PET scan, and determining a first amount of HIF-2α expression in the subject; administering to the subject an anti-ischemia treatment; administering to the subject the HIF-2α-specific radioactive tracer, subjecting the subject to a second PET scan, and determining a second amount of HIF-2α expression in the subject; and comparing the first amount and second amount of HIF-2α expression. Where the second amount of HIF-2α expression is decreased as compared to the second amount of HIF-2α expression level, there can be a decrease in the size of the ischemic area, and where the second amount of HIF-2α expression is the same or increased as compared to the second amount of HIF-2α expression level, there is no improvement in the size of the ischemic area. The HIF-2α-specific radioactive tracer can comprise an HIF-2α-specific inhibitor and a radioactive label, wherein the radioactive label is a positron emitting radioisotope

In some embodiments, the subject can have or can be at risk of developing a heart disease, an infarct, or a stroke. The ischemic area can be within a myocardium or a brain parenchyma.

In various embodiments, administering the HIF-2α-specific radioactive tracer can be by intravenous, intraarterial, or oral administration.

An embodiment provides a method of detecting pulmonary hypertension in a subject comprising administering to the subject an HIF-2α-specific radioactive tracer; subjecting the subject to a PET scan; and determining an amount of the tracer. An increased amount of the tracer as compared to a control can indicate that the subject has pulmonary hypertension.

In some embodiments, a pulmonary hypertension treatment can be administered to the subject having pulmonary hypertension.

Another embodiment provides a method of monitoring pulmonary hypertension in a subject comprising administering to a subject having pulmonary hypertension an HIF-2α-specific radioactive tracer and subjecting the subject to a first PET scan to determine a first amount of HIF-2α expression in the subject. After a period of time, an HIF-2α-specific radioactive tracer can again be administered to the subject. The subject can be administered a second PET scan. A second amount of HIF-2α expression can be determined in the subject. The first amount and second amount of HIF-2α expression can be compared to monitor pulmonary hypertension evolution in the subject over time.

In some embodiments, where the second amount of HIF-2α expression is increased as compared to the first amount of HIF-2α expression then there may be no improvement of the pulmonary hypertension in the subject, wherein where the second amount of HIF-2α expression is the same as compared to the first amount of HIF-2α expression, then there may be a stabilization of the pulmonary hypertension, and wherein where the second amount of HIF-2α expression is decreased as compared to the first amount of HIF-2α expression then there may be improvement in the pulmonary hypertension.

An embodiment provides a method of evaluating efficacy of a pulmonary hypertension treatment in a subject comprising administering to a subject having pulmonary hypertension an HIF-2α-specific radioactive tracer, subjecting the subject to a first PET scan, and determining a first amount of HIF-2α expression in the subject. The subject can then be administered a pulmonary hypertension treatment. After treatment, the subject can be administered an HIF-2α-specific radioactive tracer and a second PET scan can be administered. A second amount of HIF-2α expression in the subject can be determined. The first amount and second amount of HIF-2α expression can be compared, wherein where a second amount of the HIF-2α-specific radioactive tracer is decreased as compared to the first amount, then there may be efficacy of the pulmonary hypertension treatment.

In some embodiments, the pulmonary hypertension treatment can comprise an HIF-2α inhibitor. Where a second amount of the HIF-2α-specific radioactive tracer is equivalent or increased as compared to the first baseline level, then there may be no efficacy of the pulmonary hypertension treatment.

In various embodiments, the pulmonary hypertension can be a HIF-2α-mediated pulmonary hypertension. Administering the HIF-2α-specific radioactive tracer can be by intravenous, intraarterial, or oral administration.

An embodiment provides a method of synthesizing an HIF-2α-specific radioactive

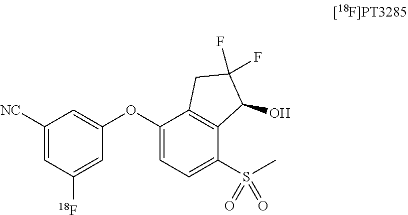 [SMILES: CS(=O)(=O)c1ccc(Oc2cc([18F])cc(C#N)c2)c2c1[C@H](O)C(F)(F)C2]

tracer having formula comprising: protecting the ketone group of a 4-fluoro-7-(methylsulfonyl)-2,3-dihydro-1H-inden-1-one in the presence of ethane-1,2-diol to form a cyclic ketal; substituting a nucleophilic aromatic group with 3-bromo-5-hydroxybenzonitrile; deprotecting a cyclic ketal group in the presence of pyridinium p-toluenesulfonate; condensing using n-butylamine; fluorinating using N-chloromethyl-N′-fluorotriethylenediammonium bis(tetrafluoroborate) and acid hydrolyzing; asymmetrically hydrogenating using RuCl(p-cymene)[(R,R)-Ts-DPEN]; performing a Pd catalytic reaction using bis(pinacolato)diboron; and labeling with [¹⁸F]KF in presence of Cu(OTf)₂. Optionally, the method can comprise protecting the hydroxyl group in the presence of MTBE before performing the Pd catalytic reaction. Optionally, the method can comprise incorporating a methoxymethyl ether (MOM) protecting group before performing the Pd catalytic reaction, and deprotecting the MOM group after radiolabeling.

Another embodiment provides a method of synthesizing an HIF-2α-specific radioactive tracer having formula

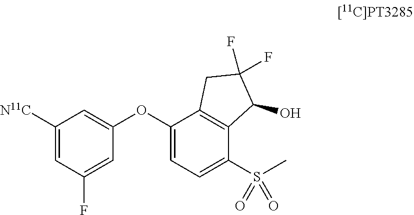

comprising protecting the ketone group of a 4-fluoro-7-(methylsulfonyl)-2,3-dihydro-1H-inden-1-one in the presence of ethane-1,2-diol to form a cyclic ketal; substituting a nucleophilic aromatic group with 3-bromo-5-fluoro-phenol; deprotecting a cyclic ketal group in the presence of pyridinium p-toluenesulfonate; condensing using n-butylamine; fluorinating using N-chloromethyl-N′-fluorotriethylenediammonium bis(tetrafluoroborate) and acid hydrolyzing; asymmetrically hydrogenating using RuCl(p-cymene)[(R,R)-Ts-DPEN]; and cyanating in the presence of Pd catalyst to produce a [¹¹C]-labeled compound.

An additional embodiment provides an automatic method for the radiosynthesis of an HIF-2α-specific radioactive tracer comprising charging a radiochemistry synthesizer with a precursor compound, mixing the precursor compound with a positron emitting radioisotope; and collecting a pure fraction containing a radiolabeled HIF-2α-specific tracer. The HIF-2α-specific radioactive tracer can be

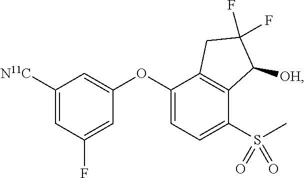

the precursor compound can be

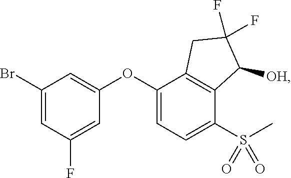 [SMILES: CS(=O)(=O)c1ccc(Oc2cc(F)cc(Br)c2)c2c1[C@H](O)C(F)(F)C2]

and the radiolabeling intermediate can be [¹¹C]HCN. The HIF-2α-specific radioactive tracer can

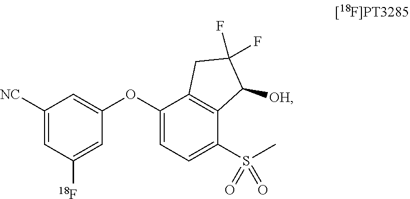 [SMILES: CS(=O)(=O)c1ccc(Oc2cc([18F])cc(C#N)c2)c2c1[C@H](O)C(F)(F)C2]

the precursor compound can be

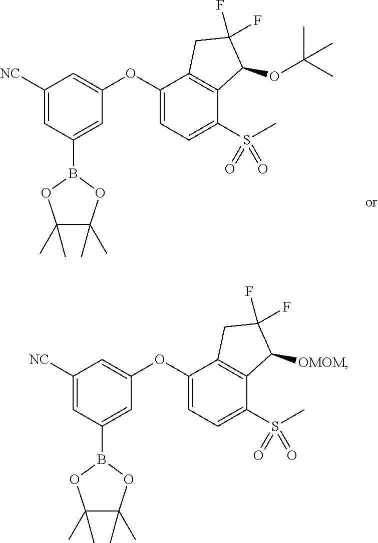

and the radiolabeling species can be [¹⁸F]fluoride.

Herein, an HIF-2α inhibitor (e.g., PT2385) is developed to be a PET tracer to specifically identify HIF-2α abnormal expression in a subject. Another embodiment involves the use of other HIF-2α binding compounds targeting the same or another pocket in HIF-2α. The approach has broad applications and can be relevant to, for example, evaluation of kidney cancer, other cancers, and ischemia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows representative tumor growth curves from a subset of patient-derived tumorgrafts illustrating sensitivity in 50% of the lines examined. Tumorgrafts with high levels of HIF-2α expression (XP164, XP374, XP469), but not those with low expression (XP462, XP490, XP530) responded to the HIF-2 inhibitor. (Blue=vehicle; green=sunitinib; red=HIF-2 inhibitor)

FIGS. 2A-B illustrate the efficacy and tolerance of PT2399 as compared to Sunitinib and vehicle. FIG. 2A shows PT2399 (red) is more effective than sunitinib (green). FIG. 2B shows PT2399 (red) is better tolerated than sunitinib (green). Experiments involved 267 mice.

FIG. 3 shows high HIF-2α expression in PT2399-sensitive but not PT2399-resistant tumors, where PT2399 is a tool compound and close analogue of PT2385 used for experiments in mice. Left: IHC for HIF-2α expression. (Scale bars, 50 μm). Right: Quantification of HIF-2α-positive cells as determined by IHC in sensitive, intermediate, and resistant tumors from all 22 tumorgraft lines (sensitive: n=10; intermediate: n=5; resistant: n=7).

FIG. 4 illustrates that PT2399 is a highly specific HIF-2α inhibitor. RNA-seq analyses identified 492 dysregulated RNAs in sensitive (HIF-2α expressing tumors) but none in resistant. Heatmap showing genes differentially regulated by PT2399 in sensitive and resistant tumors.

FIG. 5 illustrates that PT2385 shows significant activity in a phase 1 clinical trial despite multiple lines of prior therapy in most patients (4). Swimmers plot color coded by dose level (recommended phase 2 dose, 800 mg bid), with end arrows indicating ongoing therapy, and overlaid with response symbols.

FIG. 6 illustrates an acquired resistance mutation (G323E) in HIF-2α in a preclinical mouse model extensively treated with PT2399 for >6 months.

FIG. 7 illustrates ribbon diagrams of the HIF-2α PAS-B domain (pink) with PT2385 bound (left); G323E mutation (middle); and superimposition of the mutation onto HIF-2α bound PT2385 showing the clashing (right).

FIG. 8 illustrates the schematic of a possible clinical trial to evaluate (¹⁸F)PT2385 as a non-invasive tracer to measure HIF-2α expression in tumors from patients with germline mutations in the VHL gene. Green indicates signal from the tracer. PET results would be compared with tissue analyses (IHC) obtained from biopsies or tumor resections. (C1=cycle 1; C2=cycle 2; D1=Day 1).

FIG. 9 shows molecular structures of PT2385, an HIF-2α inhibitor, and [¹¹0] and [¹⁸F]-labeled PT2385, [¹¹C]PT2385 and [¹⁸F]PT2385.

FIG. 10 shows that HIF-2α binding to HIF-1β is disrupted in HEK293T cells treated with PT2385 that has been labeled with [¹¹C] ([¹¹C]PT2385; shown as PT2385 in Figure) as might be expected for PT2385 by western blot.

FIG. 11 illustrates the synthetic route to [¹¹C]PT2385.

FIG. 12 shows a diagram of TRACERlab FX M module for the automated radiosynthesis of [¹¹C]PT2385.

FIG. 13 illustrates [¹¹C]PT2385 uptake in an HIF-2α-expressing ccRCC tumorgraft. Representative PET images of XP164 tumors with 80% HIF-2α expression following [¹¹C]PT2385 administration. Representative experiment shown (n=3).

FIG. 14 illustrates [¹¹C]PT2385 uptake by HIF-2α-expressing ccRCC tumors in mice is specific and can be blocked by excess cold PT2385.

FIG. 15 illustrates an initial synthesis route to [¹⁸F]PT2385.

FIG. 16 illustrates radiochemical routes (Route B and Route C) to [¹⁸F]PT2385.

FIG. 17 shows a diagram of TRACERlab FXN pro module for the synthesis of [¹⁸F]PT2385.

FIG. 18 shows IHC for HIF-2α expression showing 5 PDX lines with high HIF-2αexpression (top row) and 5 PDX lines with low HIF-2α expression (bottom row).

FIGS. 19A-B show [¹⁸F]-PT2385 PET/CT specific detection of HIF-2α expressing human clear cell renal cell carcinoma (ccRCC) in mice. FIG. 19A shows representative [¹⁸F]-PT2385 PET/CT images of NOD/SCID mice bearing subcutaneously implanted human ccRCC tumorgrafts devoid of HIF-2α expression (XP534) (L) and HIF-2α-expressing (XP164) (R) at the designated time points following [¹⁸F]-PT2385 intravenous injection. FIG. 19B shows representative HIF-2α and CD-31 immunohistochemistry from HIF-2α the corresponding tumorgraft lines showing differences in HIF-2α expression levels and similar vascularity (based on CD-31 staining).

### DETAILED DESCRIPTION

The compositions and methods are more particularly described below, and the Examples set forth herein are intended as illustrative only, as numerous modifications and variations therein will be apparent to those skilled in the art.

Likewise, many modifications and other embodiments of the HIF-2α-specific radioactive tracer and methods described herein will come to mind to one of skill in the art having the benefit of the teachings presented in the foregoing descriptions and in the associated drawings. Therefore, it is to be understood that the methods and compositions are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art.

Overview

Adequate oxygenation requires sufficient blood flow for the proper function of organs. Insufficient blood flow, or ischemia, leads to tissue hypoxia (reduced oxygen availability) or anoxia (complete absence of oxygen). Hypoxia is a condition in which the body or a region of the body is deprived of adequate oxygen supply at the tissue level. Hypoxia may be classified as either generalized or local. Although hypoxia is often a pathological condition, variations in arterial oxygen concentrations can be part of the normal physiology, for example, during hypoventilation training or strenuous physical exercise.

Hypoxia-inducible factors (HIFs) are a group of transcription factors involved in the physiological response to oxygen concentration. The transcriptional complex HIF-1 (or HIF-2), is a heterodimer composed of an alpha and a beta subunit. The beta subunit is constitutively expressed, while the alpha subunit is, under normoxic conditions, hydroxylated and ubiquitinated by the VHL E3 ubiquitin ligase, which labels it for rapid degradation by the proteasome. In hypoxic conditions, oxygen-dependent hydroxylation is inhibited, which stabilizes the HIF complex, which can in turn upregulate several genes to promote survival in low-oxygen conditions.

Clear cell RCC (RCC or ccRCC), is the most common renal cell carcinoma type. It is characterized by the inactivation of the VHL gene (>90%) leading to the constitutive activation of HIF-1α and HIF-2α. Both HIF-1α and HIF-2α bind to HIF-1β. to form a complex (referred to as HIF-1 or HIF-2). HIF-2α in particular has been shown to play a critical role in tumorigenesis. To specifically target HIF-2, highly specific inhibitors of HIF-2α have been developed, such as PT2399, PT2385, and PT2977. HIF-2α inhibitors bind an unusual completely buried 300 Å cavity in the HIF-2α PAS-B domain leading to a conformational change and the dissociation from its obligatory partner HIF-1β, which inhibits HIF-2 ability to function as a heterodimer as required for DNA binding and transactivation.

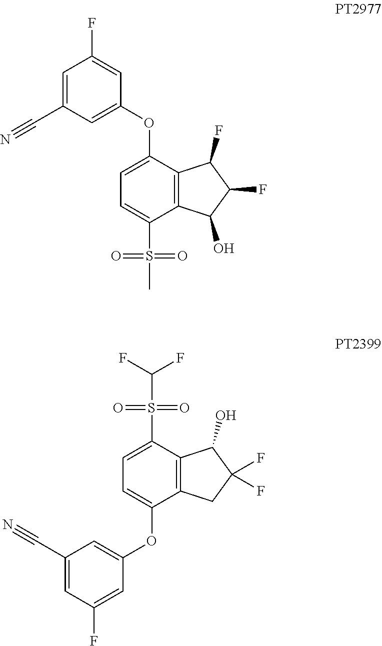 [SMILES: CS(=O)(=O)c1ccc(Oc2cc(F)cc(C#N)c2)c2c1[C@H](O)[C@H](F)[C@@H]2F ; N#Cc1cc(F)cc(Oc2ccc(S(=O)(=O)C(F)F)c3c2CC(F)(F)[C@H]3O)c1]

Provided herein are novel molecular imaging approaches to identify HIF-2α using HIF-2α-specific radioactive tracers. An HIF-2α-specific radioactive tracer comprises an HIF-2α-specific inhibitor and a positron emitting radioactive label, which can be used in various non-invasive methods, especially for detecting an HIF-2α-expressing tumors, for detecting HIF-2α inhibitor resistant tumors, for evaluating a change in HIF-2α expression in response to an anti-cancer treatment, for detecting acquisition of HIF-2α inhibitor resistance, or for detecting or monitoring an ischemic area in a subject.

Tracers

Positron-emission tomography (PET) is a non-invasive and quantitative nuclear medicine functional imaging technique with high sensitivity and specificity to observe metabolic, cellular, or molecular processes in the body by specifically designed molecular imaging probes as an aid for the diagnosis of diseases. The PET scanner detects coincidences of 511 keV gamma rays resulted from the annihilation of elections and positrons emitted from the radiolabel, which is introduced into the body on a biologically active molecule called a radioactive tracer. Different radiotracers are used for different imaging purposes, depending on what is being detected. The most common type of PET scan in standard medical care is [¹⁸F] fluorodeoxyglucose (FDG)-PET, using an analogue of glucose to visualize primary cancer and metastases, as the concentrations of FDG quantified from the tomographic images indicate the glucose consumption in regions of interest.

An HIF-2α-specific radioactive tracer can comprise an HIF-2α-specific inhibitor and a radioactive label, which can be a positron emitting radioisotope. In other embodiments it may also comprise other HIF-2α-specific compounds.

As used herein, “radioactive label” refers to a radioactive isotope, substituted for a normal atom or group in an HIF-2α inhibitor to allow its detection through medical imaging. Examples of radioisotopes include Carbon-11, ¹¹C; Iodine-123, ¹²³I; Iodine-124, ¹²⁴I; Iodine-131, ¹³¹I; Fluorine-18, ¹⁸F; Gallium-68, ⁶⁸Ga; Technetium-99m, ⁹⁹mTc; and Copper-64, ⁶⁴Cu. In an embodiment [¹¹C] (t_{1/2}=20.3 min) and [¹⁸F] (t_{1/2}=110 min), both of which are positron-emitting radioisotopes, can be incorporated into or linked to an HIF-2α-specific inhibitor without altering the structure of the molecule, and thus turning the inhibitor into a reporter that can be used to detect HIF-2α in tumors or tissues (e.g. cancerous tissues or ischemic tissues) via positron emission tomography (PET).

A radioactive tracer can be an analog of an HIF-2α-specific inhibitor, which interacts with HIF-2α in the same way as a native HIF-2α-specific inhibitor. As used herein, the term “HIF-2α-specific inhibitor” is not meant to be limited to compounds having inhibitory function against HIF-2α, but is meant to also include variants, such as chemical variants of HIF-2α-specific inhibitors that specifically bind to HIF-2α, but that may not function as HIF-2α-inhibitor. The HIF-2α-specific radioactive tracer can have the same or minimally altered structure of the HIF-2α-specific inhibitor. The radioactive tracer can be taken up by the cells of a tissue where HIF-2α is expressed, and therefore constitute a marker for such cells/tissue. As such, after injection of a radioactive tracer into a patient, a PET scanner can form tomographic images of the distribution of the tracer dynamically over the time course within the body.

HIF-2 is arguably the most important driver of clear cell renal cell carcinoma (ccRCC or RCC), which, as stated previously, is the most common renal cell carcinoma type. It is characterized by the inactivation of the VHL gene (>90%) leading to the constitutive activation of HIF-1α and HIF-2α. Both HIF-1α and HIF-2α bind to HIF-1β to form a complex (referred to as HIF-1 or HIF-2). HIF-2α in particular has been shown to play a critical role in tumorigenesis. “HIF-2α-specific inhibitor” is meant to encompass any HIF-2α-specific compound or drug that binds to HIF-2α. In an embodiment, an HIF-2α-specific inhibitor can inactivate HIF-2α. One example is PT2385, PT2399, PT2977, or other related structures binding the same pocket, which binds to the PAS-B domain, induces a conformational change, and triggers the dissociation of HIF-2α from HIF-1β. Examples of HIF-2α-specific inhibitors include, but are not limited to, PT2385, PT2399, PT2977, and other related structures binding the same pocket. In another embodiment, other HIF-2α-specific binding compounds or drugs may be used which do not necessarily inhibit HIF-2, but still specifically bind HIF-2α.

In an embodiment, a radioactive tracer comprises an HIF-2α-specific inhibitor that is covalently linked or otherwise bound or linked to a radioisotope.

Methods of Detecting HIF-2α-Expressing Cells and Tumors

In all cases described herein an amount or level HIF-2α expression or an amount or level of HIF-2α-specific radioactive tracer can be determined at a region or regions of interest in a subject. A region or regions of interest can be, for example, a tumor, an area in which a tumor is located, a tissue, a portion of a tissue, an organ (e.g., kidney, pancreas), a portion of an organ (e.g., a lobe of a lung, a heart chamber, a heart valve, or a portion of a kidney), an area in which an organ is located, an ischemic area or region, a limb (e.g., a hand, foot, arm, or leg), the brain, a portion of the brain, or a portion of a body (e.g., respiratory tract, abdomen, etc.). A health professional will be able to determine a suitable region or regions of interest of a subject.

An embodiment provides a method of detecting an HIF-2α-expressing tumor in a subject comprising: administering to a subject having a tumor an HIF-2α-specific radioactive tracer as described herein; subjecting the subject to a PET scan; and determining the amount of the HIF-2α-specific radioactive tracer in the tumor at, e.g., regions of interest. An increased amount of the tracer as compared to a control (such as other tissues used as baseline or tissue from other healthy subjects) indicates an HIF-2α-expressing tumor. The PET scan can be performed dynamically immediately after the administration of the HIF-2α-specific radioactive tracer out to 4 hours post-injection or static at any point in between (0-4 hours). Where an HIF-2α-expressing tumor is detected, an appropriate treatment can be prescribed (e.g., an HIF-2α inhibitor alone or in combination with other therapies). Where a non-HIF-2α-expressing tumor is detected, an appropriate treatment can be prescribed (e.g., therapies other than HIF-2α inhibitors).

Cancer is a group of diseases involving abnormal cell growth with the potential to invade or spread to other parts of the body. The term “cancer” refers to a group of diseases characterized by abnormal and uncontrolled cell proliferation starting at one site (primary site) with the potential to invade and to spread to others sites (secondary sites, metastases) which differentiate cancer (malignant tumors) from benign tumors. Virtually all the organs can be affected, leading to more than 100 types of cancer that can affect humans. Cancers can result from many causes including genetic predisposition, viral infection, exposure to ionizing radiation, exposure environmental pollutants, tobacco and/or alcohol use, obesity, poor diet, lack of physical activity, or any combination thereof. The most common types of cancer in males are lung cancer, prostate cancer, and colorectal cancer. In females, the most common types are breast cancer, colorectal cancer, and lung cancer. As used herein, “neoplasm” or “tumor,” including grammatical variations thereof, means new and abnormal growth of tissue, which may be benign or cancerous, and can include both primary tumors and metastases. In a related aspect, the neoplasm is indicative of a neoplastic disease or disorder, including but not limited to, various cancers.

While the present methods are particularly applicable to clear cell renal cell carcinoma, the methods can be applied to any cancer type. Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; and Wilms' Tumor.

The term “subject” as used herein refers to any individual or patient to which the methods described herein are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals (including cows, horses, goats, sheep, pigs, chickens, etc.), and primates (including monkeys, chimpanzees, orangutans and gorillas) are included within the definition of subject.

The terms “administration of” and or “administering” means providing a tracer composition to a subject. Administration routes for a tracer can be by any route, including, for example, enteral, topical, or parenteral. As such, administration routes include but are not limited to intravenous, intraperitoneal, intraarterial, intrathecal, intracapsular, intraorbital, intracardiac, transtracheal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

A PET scan can be used to determine the amount of positron emitting radiotracer taken up by cells. Standardized uptake value, SUV, (also referred to as the dose uptake ratio, DUR) is a widely used, robust PET quantifier, calculated simply as a ratio of tissue radioactivity concentration (for example in units [kBq/mL]) at time T, C_{PET}(T), and administered dose (for example in units [MBq]) at the time of injection divided by body weight (BW, usually in units [kg]).

SUV_{Bw}=C_{PET}(T)/(Dose/Weight)

Tissue radioactivity and dose can be decay corrected to the same time point. Instead of the body weight, the administered dose can also be corrected by the lean body mass (LBM), or body surface area (BSA). The imaging should take place at a late time point, and at the same time point, if results are to be compared.

When assessed by PET, cancer treatment response can be assessed by calculating the SUV on the highest image pixel in the tumor regions (SUVₘₐₓ), because this provides lower inter-observer variability than averaged SUV (SUVₘₑₐₙ).

In animal studies, dissected tissue samples can be weighted and radioactivity measured at the tumor site. Radioactivity can then be divided by sample weight to calculate the concentration (Bq/g). With injected dose and animal weight the SUV could be calculated similarly as from PET data. However, in animal studies the animal weight is often not taken into account: radioactivity concentration is simply divided by injected dose and multiplied by 100, and outcome is percent of injected dose per gram of tissue (% ID/g). Similar calculation can be done to PET data. In PET image the radioactivity concentration is measured per tissue volume (Bq/mL) instead of mass, and therefore the outcome will be in units % ID./mL or % ID/L. If tissue density (g/mL) is known or assumed to be 1 g/mL, it can be converted to % ID/g.

An HIF-2α specific radioactive tracer comprises an HIF-2α inhibitor; therefore, the radioactive tracer, like the inhibitor, is taken up by the cells of the subject to whom the tracer is administered. The tracer can interact with HIF-2α in cells that express it. When the PET scan is performed, and the amount of the tracer is measured (SUV value), it can be compared to a control value.

As used herein, a “control” can refer a value, such as a SUV value, measured in a tissue where HIF-2α is not abnormally expressed. As HIF-2α expression is highly controlled and dependent on oxygen concentration in the tissue, a control value, such as an SUV value, can be a value measured in a subject that does not have a condition that induces the expression of HIF-2α (e.g., a tumor), a value measured in a same subject but in an area that is free from the condition (e.g., tumor free area in a subject having a tumor elsewhere), or a composite control value obtained from a plurality of control non-diseased samples. Therefore, by comparing the amount of the tracer in a tumor to a control value, the methods can indicate the presence of an HIF-2α-expressing tumor in a subject. A control value can also be a value obtained from the same patient at an earlier time point.

As a tumor grows, rapidly proliferating tumor cells consume available oxygen, which create hypoxic micro-environments in solid tumors, where oxygen concentration is significantly lower than in healthy tissues. Under those hypoxic conditions, the oxygen-dependent hydroxylation of the alpha subunit of HIFs is inhibited, which stabilizes the HIF complex, which can in turn upregulate several genes to promote survival in low-oxygen conditions. In ccRCC, where VHL mutations are predominant, under normoxic conditions, the alpha subunit of HIFs is hydroxylated, but in the absence of a functional VHL protein there is no ubiquitination and consequent degradation of the alpha subunit, which is available to form an HIF complex with the beta subunit. HIF-2α has been shown to be more involved in tumorigenesis than HIF-1α; therefore, tumors carrying a VHL mutation (such as ccRCCs) or those with a hypoxic-microenvironment can abnormally express HIF-2α. As used herein “HIF-2α-expressing tumor” refers to both tumors with hypoxic-microenvironment and tumors (such as ccRCCs) carrying a VHL mutation in which all or isolated areas of the tumor can express HIF-2α.

When the amount of tracer measured by PET scan in the tumor of a subject is higher than a control value, it can indicate that the tumor as a whole or that some areas in the tumor express HIF-2α. However, when the amount of tracer measured in a tumor of a subject is lower or equivalent to the control value, it can indicate that the tumor does not express HIF-2α or is expressing HIF-2α at a lower level (e.g., expressed at a lower level due to treatment).

As further exemplified below, the detection of an HIF-2α-expressing tumor in a subject by PET scan using an HIF-2α radiotracer can be used to identify patients that are likely to respond to an anti-cancer treatment that comprises an HIF-2α inhibitor. That is, patients with a tumor or cancerous cells that express HIF-2α ata level higher than a control can be responsive to treatment with an HIF-2α inhibitor.

When the amount of tracer measured by PET scan in the tumor of a subject is higher than a control value, the tumor as a whole or some areas in the tumor express HIF-2α, and therefore, it is likely that the tumor would be responsive to an HIF-2α inhibitor treatment. However, when the amount of tracer measured in a tumor of a subject is lower or equivalent to the control value, the tumor does not express HIF-2α, and is not likely to be responsive to an HIF-2α inhibitor treatment. Regardless of the likelihood of responsiveness to an HIF-2α inhibitor treatment, a tumor can be treated by any conventional anti-cancer treatment available, including by an HIF-2α inhibitor treatment, as evaluated appropriate by a physician.

The term “treatment” is used interchangeably herein with the term “therapeutic method” and refers to both 1) therapeutic treatments or measures that cure, slow down, lessen symptoms of, and/or halt progression of a diagnosed pathologic condition or disorder, and 2) and prophylactic/preventative measures. Those in need of treatment can include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor, or other clinician. Generally, the response is either amelioration of symptoms in a patient or a desired biological outcome.

As used herein, “anti-cancer treatment” can include any method that can be used to treat, ameliorate, or lessen the symptoms of cancer or a tumor or that can reduce the amount or cancerous cells or the amount or size of tumors. Treatments can include surgery, radiotherapy, chemotherapy, targeted therapy, immunotherapy, or any combination thereof. In some aspects, administration can be in combination with one or more additional therapeutic agents. The phrases “combination therapy”, “combined with” and the like refer to the use of more than one treatment simultaneously to increase the response. Such therapies can be administered prior to, simultaneously with, or following administration of one another.

The term “chemotherapy” or “chemotherapeutic agent” as used herein refers to any therapeutic agent used to treat cancer. Examples of chemotherapeutic agents include, but are not limited to, (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising Fl inhibitor (Tipifarnib), CDK inhibitors (Abemaciclib, Alvocidib, Palbociclib, Ribociclib, and Seliciclib), Prl inhibitor (Bortezomib, Carfilzomib, and Ixazomib), Phl inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

The term “immunotherapy” refers to any type of therapy that ameliorates, treats, or prevents a malignancy in a subject by assisting or boosting the subject's immune system in eradicating cancerous cells. Modulating the immune system includes inducing, stimulating, or enhancing the immune system as well as reducing, suppressing, or inhibiting the immune system. Immunotherapy can be active or passive. Passive immunotherapy relies on the administration of drugs, such as monoclonal antibodies directed against the target to eliminate it. For example, tumor-targeted monoclonal antibodies have demonstrated clinical efficacy to treat cancer. Active immunotherapy aims to induce cellular immunity and establish immunological memory against the target agent. Active immunotherapy includes, but is not limited to, vaccination, and immune modulators.

Types of immunotherapy include, for example, immune checkpoint inhibitors, T-cell transfer therapy (i.e., adoptive cell therapy, adoptive immunotherapy, or immune cell therapy), monoclonal antibodies (e.g., monoclonal antibodies that can mark cancer cells so that they will be better identified and destroyed by the immune system), treatment vaccines (e.g., Sipuleucel-T, T-VEC), and immune system modulators [e.g., cytokines, interferons, interleukins (e.g., IL-2; IL-11), granulocyte-macrophage colony-stimulating factor (GM-CSF) and granulocyte colony-stimulating factor (G-CSF), BCG], and immunomodulatory drugs such as thalidomide, lenalidomide, pomalidomide, imiquimod.

“Checkpoint inhibitor therapy” is a form of cancer treatment that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Targeted therapies (also called targeted cancer therapies herein) are drugs or other substances (e.g., siRNA) that block the growth and spread of cancer by interfering with specific molecular targets that are involved in the growth, progression, and spread of cancer. Targeted therapies differ from standard chemotherapy in several ways including that they act on specific molecular targets that are associated with cancer, whereas most standard chemotherapies act on all rapidly dividing normal and cancerous cells. Examples of a targeted therapies include HIF-2α inhibitors such as PT2385, PT2399, PT2977, or other related structures binding the same pocket.

For ccRCC specifically, available treatment options include, surgery, radiotherapy, immune checkpoint inhibitors combined with targeted therapies, such as anti-angiogenic targeted therapies. Targeted therapies for, e.g., RCC can include pembrolizumab plus axitinib, or avelumab plus axitinib; anti-vascular endothelial growth factor (VEGF) or VEGF receptor inhibitors, such as sunitinib, bevacizumab, pazopanib; cabozantinib or sorafenib; mammalian target of rapamycin (mTOR) inhibitors, such as temsirolimus, or everolimus; immune checkpoint inhibitors such as ipilimumab plus nivolumab; cytokine therapy, such as interferon-alpha and interleukin-2 (IL-2); or combination thereof.

Methods of Detecting HIF-2α Inhibitor Resistant Tumors

An embodiment provides methods of detecting an HIF-2α inhibitor resistant tumor in a subject comprising administering to a subject having a tumor an HIF-2α-specific radioactive tracer; subjecting the subject to a PET scan; and determining the amount of the HIF-2α-specific radioactive tracer at, e.g., regions of interest, wherein a low amount of tracer indicates an HIF-2α inhibitor resistant tumor. The PET scan can be performed dynamically immediately after the administration of the HIF-2α-specific radioactive tracer out to 4 hours post-injection or static at any time points in between (0-4 hours).

As further detailed in the examples below, tumors are highly plastic, and the administration of a drug applies a selective pressure that is ultimately evaded through mutation and resistance to the drug. A radioactive tracer described herein can be used to detect tumors that are or that become HIF-2α inhibitor resistant over time.

When the amount of tracer measured by PET scan in the tumor of a subject is lower or equivalent to a control value, it can indicate that the tumor does not express HIF-2α. The tumor is then not likely to be responsive to an HIF-2α inhibitor treatment. In such case, the patient can be treated by anti-cancer therapies other than HIF-2α inhibitors, as the tumor is not likely to respond to the inhibitors.

Methods of Evaluating a Change in HIF-2α Expression

An embodiment provides methods of evaluating a change in HIF-2α expression in a subject in response to an anti-cancer treatment. The methods can comprise administering to a subject having a tumor an HIF-2α-specific radioactive tracer and subjecting the subject to a first PET scan. The PET scan can be performed dynamically immediately after the administration of the HIF-2α-specific radioactive tracer out to 4 hours post-injection or static at any time points in between (0-4 hours). A first amount of HIF-2α expression can be determined at, e.g., regions of interest. The subject can be administered an anti-cancer treatment. At this point, the anti-cancer treatment can be administered 1, 2, 3, 4 or more times a day for 1, 2, 5, 7, 14, 21, 28 days, or 1, 2, 3, 4, 5, 6, or more months before the next step. When ready to proceed with the method, the subject is administered an HIF-2α-specific radioactive tracer as described herein. The subject can be subjected to a second PET scan, and a second amount of HIF-2α expression at, e.g., the same one or more regions of interest can be determined. The PET scan can be performed dynamically immediately after the administration of the HIF-2α-specific radioactive tracer out to 4 hours post-injection or static at any time points in between (0-4 hours). The first amount and second amount of HIF-2α expression can be compared.

Where the drug is a cancer drug intended to reduce HIF-2α protein expression levels, such as an siRNA, and second amount of tracer measured by the second PET scan of the tumor of a subject is lower than the first amount of tracer measured by the first PET scan of the tumor of the subject, then efficacy of the treatment is likely.

However, when the second amount of tracer measured by a second PET scan in the tumor of a subject is the same or higher than the first amount of tracer measured by the first PET scan of the tumor of the subject, then efficacy of the cancer drug intended to reduce HIF-2α protein expression levels treatment is not likely. In this case, an alternative therapy may be considered.

Several lines of anti-cancer therapies can be administered to a subject over the course of the treatment of a cancer. Each anti-cancer therapy can have individual effect on tumor characteristics and on its responsiveness to an HIF-2α inhibitor therapy. Accordingly, the determination of a change in HIF-2α expression can be assessed after the administration of an anti-cancer treatment, whether or not it is the first to be administered to the subject, and as many times as required to follow the changes of HIF-2α expression over time. The anti-cancer therapy can be adjusted as needed based on the changes in the HIF-2α inhibitor sensitivity over time.

Therefore, the steps of administering to the subject an HIF-2α-specific radioactive tracer; subjecting the subject to a PET scan, and determining a second amount of HIF-2α expression in the subject; and comparing the first amount and second amount of HIF-2α expression can be repeated after each anti-cancer treatment regimen. Additionally, in cases of repeated measures, the latest amount of tracer measured by PET scan in the tumor of a subject can be compared to any one or to all of the previously measured amounts to evaluate the changes over time and in response to the various anti-cancer therapies.

An embodiment provides a method of evaluating efficacy of an HIF-2α depletion therapy in a subject. The method comprises administering an HIF-2α-specific radioactive tracer to the subject and subjecting the subject to a first PET scan. The PET scan can be performed dynamically immediately after the administration of the HIF-2α-specific radioactive tracer out to 4 hours post-injection or static at any time points in between (0-4 hours). A first baseline level of the HIF-2α-specific radioactive tracer can be determined. The baseline level can be determined at, e.g., a region or regions of interest. The subject can be administered an HIF-2α depletion therapy, for example siRNA therapy. At this point, the HIF-2α depletion therapy can be administered 1, 2, 3, 4 or more times a day for 1, 2, 5, 7, 14, 21, 28 days, or 1, 2, 3, 4, 5, 6, or more months before the next step. When ready to proceed with the method, the subject is administered an HIF-2α-specific radioactive tracer as described herein. The subject can be subjected to a second PET scan. The PET scan can be performed dynamically immediately after the administration of the HIF-2α-specific radioactive tracer out to 4 hours post-injection or static at any time points in between (0-4 hours). A second level of the HIF-2α-specific radioactive tracer can be determined at, e.g., the same region or regions of interest. The second level can be compared to the first baseline level. Where a second level of the HIF-2α-specific radioactive tracer is decreased as compared to the first baseline level, then there is efficacy of an HIF-2α depletion therapy.

As used herein, the term “HIF-2α depletion therapy” refers to any therapy that can reduce or limit HIF-2α expression in a tissue, for example in a tumor. An HIF-2α depletion therapy can reduce or limit the amount of HIF-2α expression by reducing or limiting HIF-2α gene transcription, HIF-2α translation, and/or by inducing the active elimination of HIF-2α protein. Non-limiting examples of HIF-2α depletion therapy include siRNA therapy, shRNA therapy, miRNA therapy, and neutralizing agents.

The HIF-2α depletion therapy can comprise an siRNA targeting HIF-2α. Where a second level of the HIF-2α-specific radioactive tracer is equivalent or increased as compared to the first baseline level, then there is an acquisition of resistance to the HIF-2α depletion therapy, characterized by a restoration of HIF-2α levels.

The efficacy of HIF-2α depletion therapy, such as siRNA therapy, requires the presence of specific receptors on the surface of the tumor cells, whose downregulation can prevent siRNA entry into tumor cells, leading to HIF-2α accumulation. In such case, an HIF-2α accumulation can be detected in the PET scan, as a restoration of HIF-2α levels despite siRNA therapy.

An siRNA targeting HIF-2α can include for example ARO-HIF2 (Arrowhead Pharmaceuticals), a drug for the treatment of ccRCC. ARO-HIF2 is designed to inhibit the production of HIF-2α, which has been linked to tumor progression and metastasis in ccRCC.

HIF2 RNAi selectively targets and silences HIF2α expression, using a proprietary targeted-RNAi molecule (TRiM™ ) delivery platform for the treatment of ccRCC. The TRiM™ based HIF2 construct comprises a highly potent RNAi trigger using stabilization chemistries, targeting ligands to facilitate delivery, and structures to enhance pharmacokinetics (PK). HIF2 RNAi can silence HIF2α mRNA (85% knock-down) resulting in tumor growth inhibition in mouse xenograft models. Significant improvement in overall survival was also seen in a patient derived xenograft model. Histology evaluation of tumor samples revealed extensive tumor destruction with clusters of apoptotic cells and necrosis. Loading doses can be administered four hours apart without loss in potency.

ARO-HIF2 is an attractive target for intervention because over 90% of ccRCC tumors express a mutant form of the Von Hippel-Landau protein that is unable to degrade HIF-2α, leading to its accumulation during tumor hypoxia and promoting tumor growth. ARO-HIF2 is delivered using a new extra-hepatic delivery vehicle. ARO-HIF2 is currently tested in a Phase 1b clinical trial to evaluate the safety and efficacy of ARO-HIF2 injection, and to determine the recommended Phase 2 dose in the treatment of patients with advanced ccRCC.

Methods of Detecting Acquisition of Resistance

Provided herein are methods of detecting acquisition of HIF-2α inhibitor resistance in a subject.

Anti-cancer therapy resistance occurs when cancers that have been responding to a therapy suddenly stop to do so and begin to grow, i.e., the cancer cells are resisting the effects of the drug. Resistance can occur when some of the cells that are not killed by the therapy mutate and become resistant to the drug. Alternatively, the cancer cells can pump the drug out of the cell, mutate to render the transporter that facilitates drug entry in the cell inoperable, or may develop a mechanism that inactivates the drug. Because of the development of drug resistance, drugs are often given in combination, which may reduce the incidence of developing resistance to any one drug.

When a tumor expresses HIF-2α, it may be sensitive to an HIF-2α inhibitor, and the response of the tumor to the treatment may be monitored over time. When the amount of tracer measured by a PET scan in an HIF-2α inhibitor sensitive tumor is reduced over time, it can indicate that the tumor as a whole or that some areas in the tumor that previously expressed HIF-2α do not express it anymore or that less cancer cells are HIF-2α inhibitor sensitive. Accordingly, some cancer cells may have developed a resistance to the treatment. Resistance can also arise as a consequence of mutations in HIF-2α that block drug (and tracer) binding, as in the case of PT2385 (or other similar drugs binding to the same pocket as the tracer whose binding would be blocked by acquisition of a resistance mutation).

Results can be confirmed by sequencing of the HIF-2α gene. The detection of mutations can indicate acquisition of resistance.

In an embodiment a method of detecting acquisition of HIF-2α inhibitor resistance in a subject is provided. A subject is administered an HIF-2α-specific radioactive tracer as described herein. The subject is subjected to a first positron emission topography (PET) scan. The PET scan can be performed dynamically immediately after the administration of the HIF-2α-specific radioactive tracer out to 4 hours post-injection or static at any time points in between (0-4 hours). A first baseline level of the HIF-2α-specific radioactive tracer can be determined. The subject can then be administered an HIF-2α inhibitor as described herein. At this point, the HIF-2α inhibitor can be administered 1, 2, 3, 4 or more times a day for 1, 2, 5, 7, 14, 21, 28 days, or 1, 2, 3, 4, 5, 6, or more months before the next step. When ready to proceed with the method, the subject is administered an HIF-2α-specific radioactive tracer as described herein. The subject is then subjected to a second PET scan. A second level of the HIF-2α-specific radioactive tracer is determined. The PET scan can be performed dynamically immediately after the administration of the HIF-2α-specific radioactive tracer out to 4 hours post-injection or static at any time points in between (0-4 hours). The second level is compared to the first baseline level. Where a second level of the HIF-2α-specific radioactive tracer is decreased as compared to the first baseline level, then there can be an acquisition of HIF-2α inhibitor resistance.

It is important to note that differences observed between a first and a second level of the HIF-2α-specific radioactive tracer determined by PET scan can be interpreted differently based on the treatment received by the subject between the first and the second PET scan. For example, when a subject is administered an HIF-2α inhibitor, a decreased second level of the HIF-2α-specific radioactive tracer can indicate a reduced amount or the absence of HIF-2α in the tumor. This can indicate an acquisition of HIF-2α inhibitor resistance, for example through the acquisition of a somatic HIF-2α mutation in the tumor.

In contrast, when a subject is administered an HIF-2α depletion therapy, such as a siRNA therapy for example, a decreased second level of the HIF-2α-specific radioactive tracer can indicate reduction or the absence of HIF-2α in the tumor, as the result of an efficient HIF-2α depletion in the tumor. This can indicate the efficacy of an HIF-2α depletion therapy.

Such evaluation of the amount of tracer measured in an HIF-2α inhibitor sensitive tumor can be assessed multiple times during the course of the treatment. Accordingly, the steps of administering to the subject the HIF-2α-specific radioactive tracer, subjecting the subject to a PET scan, determining a level of the HIF-2α-specific radioactive tracer in the tumor or region of interest and comparing the level to a reference (e.g., a baseline measurement or a previously measured value or amount), can be repeated as often as considered appropriate by a physician, to closely monitor the acquisition of an HIF-2α inhibitor resistance.

Methods of Detecting/Monitoring Ischemic Areas

lschemia is a restriction in blood supply to tissues, causing a shortage of oxygen that is needed for cellular metabolism; it is generally caused by problems with blood vessels, with resultant damage to or dysfunction of tissue. It can be partial (poor perfusion) or total. Without immediate intervention, ischemia may progress quickly to tissue necrosis and gangrene within a few hours. lschemia can affect, e.g., the heart, the brain, the intestines, the skin or limbs. Cardiac ischemia occurs when the heart muscle, or myocardium, receives insufficient blood flow; it most frequently results from atherosclerosis. Large and small intestines can be affected by ischemia, which may result in an inflammatory process such as ischemic colitis or mesenteric ischemia. Brain ischemia (or stroke) is the insufficient blood flow to the brain. Acute ischemic stroke is a neurologic emergency that may be reversible if treated rapidly. lschemia is a vascular disease involving an interruption in the arterial blood supply to a tissue, organ, or extremity that, if untreated, can lead to tissue death. It can be caused by embolism, thrombosis of an atherosclerotic artery, or trauma. In highly metabolically active tissues such as the heart and brain, irreversible damage to tissues can occur in as little as 3-4 minutes in ischemic conditions. It is thus very important to be able to monitor subjects having ischemic episodes using non-invasive methods.

The treatment options, or “anti-ischemia treatments” include injection of an anticoagulant, thrombolysis, embolectomy, surgical revascularization, or partial amputation. Anticoagulant therapy is initiated to prevent further enlargement of the thrombus. Thrombolytic agents, such as recombinant tissue plasminogen activator (tPA), streptokinase, or urokinase can be administered to resolve the clot. Direct arteriotomy may be necessary to remove the clot. Surgical revascularization may be used in the setting of trauma (e.g., laceration of the artery). Amputation is reserved for cases where limb salvage is not possible.

Provided herein are methods of detecting an ischemic area in a subject. The method comprises administering an HIF-2α-specific radioactive tracer to the subject and subjecting the subject to a PET scan. The PET scan can be performed dynamically immediately after the administration of the HIF-2α-specific radioactive tracer out to 4 hours post-injection or static at any time points in between (0-4 hours). An amount of the HIF-2α-specific radioactive tracer can be determined. The amount of tracer can be determined in a region or regions of interest. The amount can be compared a level in reference area. Where an amount of the tracer is increased as compared to a reference area, it indicates ischemia.

Treatment can be prescribed where an ischemic area is detected. A level in a reference area (or control) can be, for example, a value obtained from non-ischemic tissues of the patient or a value obtained from non-ischemic tissues of one or more control tissues from other patients that do not have ischemia.

An embodiment provides methods of monitoring an ischemic area in a subject. The method comprises administering to a subject suspected of having an ischemic area an HIF-2α-specific radioactive tracer and subjecting the subject to a first PET scan. The PET scan can be performed dynamically immediately after the administration of the HIF-2α-specific radioactive tracer out to 4 hours post-injection or static at any time points in between (0-4 hours). A first amount of HIF-2α expression at, e.g., the regions of interest can be determined. The subject can then be administered an anti-ischemia treatment. At this point, the anti-ischemia treatment can be administered 1, 2, 3, 4 or more times a day for 1, 2, 5, 7, 14, 21, 28 days, or 1, 2, 3, 4, 5, 6, or more months before the next step. When ready to proceed with the method, the subject is administered an HIF-2α-specific radioactive tracer as described herein. The subject can be subjected to a second PET scan. The PET scan can be performed dynamically immediately after the administration of the HIF-2α-specific radioactive tracer out to 4 hours post-injection or static at any time points in between (0-4 hours). A second amount of HIF-2α expression can be determined in the subject at, e.g., the same region or regions of interest. The second amount can be compared to the first amount. Where the second amount of HIF-2α expression is decreased as compared to the first amount of HIF-2αthen there is a decrease in size of the ischemic area. Where the second amount of HIF-2α expression is the same or increased as compared to the first amount of HIF-2α expression, then there is no improvement in the size of the ischemic area.

When the first amount of tracer measured by a first PET scan in an ischemic area of a subject is higher than a control value, as measured in a tissue where HIF-2α is not expressed, or in a non-ischemic area, it can indicate that the area wholly or partially expresses HIF-2α, at an ischemic level. In such case, the patient can be treated by an anti-ischemic therapy.

In such case, after an anti-ischemia therapy, a second amount of tracer measured by a second PET scan in the ischemic area of a subject can be evaluated.

1) when the second amount of tracer measured by a second PET scan in the ischemic area of a subject is higher or about the same than the first amount, as measured in the area prior to the anti-ischemia treatment, it can indicate that the treatment was not successful.

2) when the second amount of tracer measured by a second PET scan in the ischemic area of a subject is lower than the first amount, as measured in the tissue prior to the anti-ischemic treatment, it can indicate that the treatment was successful.

Methods of Detecting/Monitoring Pulmonary Hypertension

Pulmonary hypertension (PH) is a condition characterized by an increased blood pressure within the arteries of the lungs. The cause is often unknown, and symptoms can include shortness of breath, syncope, tiredness, chest pain, swelling of the legs, and a fast heartbeat. According to the World Health Organization, pulmonary hypertensions are classified into five groups, based on cause. Furthermore, a distinction can be made between primary PH (resulting from a disease of the pulmonary arteries) and secondary PH (resulting secondary to other, non-vascular causes). Specifically, PH can be classified as WHO Group I—Pulmonary arterial hypertension (PAH), WHO Group II—Pulmonary hypertension secondary to left heart disease, WHO Group III—Pulmonary hypertension due to lung disease, chronic hypoxia, WHO Group IV—chronic arterial obstruction, or WHO Group V—Pulmonary hypertension with unclear or multifactorial mechanisms.

In pulmonary hypertension due to lung diseases and/or hypoxia (WHO Group 3), low levels of oxygen in the alveoli (due to respiratory disease or living at high altitude) cause constriction of the pulmonary arteries. This phenomenon is called hypoxic pulmonary vasoconstriction and it is initially a protective response designed to stop too much blood flowing to areas of the lung that are damaged and do not contain oxygen. When the alveolar hypoxia is widespread and prolonged, this hypoxia-mediated vasoconstriction occurs across a large portion of the pulmonary vascular bed and leads to an increase in pulmonary arterial pressure, with thickening of the pulmonary vessel walls contributing to the development of sustained pulmonary hypertension. Prolonged hypoxia also induces HIF-1α and HIF-2α , which directly activates downstream growth factor signaling that causes irreversible proliferation and remodeling of pulmonary arterial endothelial cells, leading to chronic pulmonary arterial hypertension.

Other pathologies, such as polycythemias which can result from multiples causes including mutations in proteins involves in oxygen sensing and erythropoiesis can lead to the development of PH. For example, certain mutations in the VHL (von Hippel-Lindau) gene, such as those responsible for Chuvash polycythemia can lead to the development of PH due to the upregulation of endothelin-1 or HIF-2α.

In various embodiments, the methods described herein allow the detection and monitoring of HIF-2α-mediated PH, that is, any form of PH that can be caused by chronic hypoxia, resulting in elevated levels of HIF-2α. Polycythemia can also be detected and monitored in the same manner.

Pulmonary Hypertension Treatments

There is currently no cure for PH. Treatment mainly depends on the type of disease, and can include supportive measures such as oxygen therapy, diuretics, and medications to inhibit blood clotting. Medications specifically used to treat PH include endothelin-1 receptor antagonists, HIF-2α inhibitors, epoprostenol, treprostinil, iloprost, bosentan, ambrisentan, macitentan, sildenafil, and lung transplantation in the most severe cases.

In various embodiments, the pulmonary hypertension treatment can comprise an HIF-2α inhibitor such as MK-6482, PT2385, PT2399, or other related structures binding the same pocket.

Provided herein are methods of detecting pulmonary hypertension in a subject. The method comprises administering to the subject an HIF-2α-specific radioactive tracer and subjecting the subject to a PET scan. The PET scan can be performed dynamically immediately after the administration of the HIF-2α-specific radioactive tracer out to 4 hours post-injection or static at any time points in between (0-4 hours). An amount of HIF-2α-specific radioactive tracer can be determined. The amount of tracer can be determined in a region or regions of interest (e.g., lungs). The amount can be compared a level in a reference or control. Where an amount of the tracer is increased as compared to a control, it indicates that the subject has pulmonary hypertension.

A level in a reference area (or control) can be, for example, a value obtained from pulmonary tissues of one or more control tissues from other patients that do not have pulmonary hypertension. Treatment can be prescribed where pulmonary hypertension is detected.

An embodiment provides a method of monitoring pulmonary hypertension in a subject. The method comprises administering to a subject having pulmonary hypertension an HIF-2α-specific radioactive tracer and subjecting the subject to a first PET scan. The first PET scan can be performed dynamically immediately after the administration of the HIF-2α-specific radioactive tracer out to 4 hours post-injection or static at any time points in between (0-4 hours). A first amount of HIF-2α expression at one or more regions of interest (e.g., lungs) can be determined. After a period of time following the determination of the first amount of HIF-2α expression (e.g., about 1, 7, 14, 21, 20, 60, 90 days or more), and when ready to proceed with the method, the subject can be administered an HIF-2α-specific radioactive tracer as described herein. The subject can be subjected to a second PET scan. The second PET scan can be performed dynamically immediately after the administration of the HIF-2α-specific radioactive tracer out to 4 hours post-injection or static at any time points in between (0-4 hours). A second amount of HIF-2α expression can be determined in the subject at, e.g., the same region or regions of interest (e.g., lungs). The second amount can be compared to the first amount to monitor pulmonary hypertension evolution in the subject over time. Where the second amount of HIF-2α expression is increased as compared to the first amount of HIF-2α expression, there may have been no improvement of the pulmonary hypertension in the subject during the time period between the two assessments. Where the second amount of HIF-2α expression is the same as compared to the first amount of HIF-2α expression, there may be a stabilization of the pulmonary hypertension. Where the second amount of HIF-2α expression is decreased as compared to the first amount of HIF-2α expression there may have been an improvement in pulmonary hypertension in the subject during the time period between the two assessments.

Another embodiment provides a method of evaluating efficacy of a pulmonary hypertension treatment in a subject. The method comprises administering to a subject having pulmonary hypertension an HIF-2α-specific radioactive tracer and subjecting the subject to a first PET scan. The first PET scan can be performed dynamically immediately after the administration of the HIF-2α-specific radioactive tracer out to 4 hours post-injection or static at any time points in between (0-4 hours). A first amount of HIF-2α expression at one or more regions of interest (e.g., lungs) can be determined. The subject can then be administered a pulmonary hypertension treatment. At this point, the pulmonary hypertension treatment can be administered 1, 2, 3, 4 or more times a day for 1, 2, 5, 7, 14, 21, 28 days, or 1, 2, 3, 4, 5, 6, or more months before the next step. When ready to proceed with the method, the subject can be administered an HI F-2α-specific radioactive tracer as described herein. The subject can be subjected to a second PET scan. The second PET scan can be performed dynamically immediately after the administration of the HIF-2α-specific radioactive tracer out to 4 hours post-injection or static at any time points in between (0-4 hours). A second amount of HIF-2α expression can be determined in the subject at the same region or regions of interest. The second amount can be compared to the first amount. Where a second amount of the HIF-2α-specific radioactive tracer is decreased as compared to the first amount there may be efficacy of the pulmonary hypertension treatment. Where the second amount of HIF-2α expression is equivalent or increased as compared to the first amount of HIF-2α expression there may be a lack of efficacy of the pulmonary hypertension treatment in the subject.

The HI F-2α-specific radioactive tracer can be administered by intravenous, intraarterial, or oral administration.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1. Characterization of Tumors Responsive to HIF-2 Inhibitor

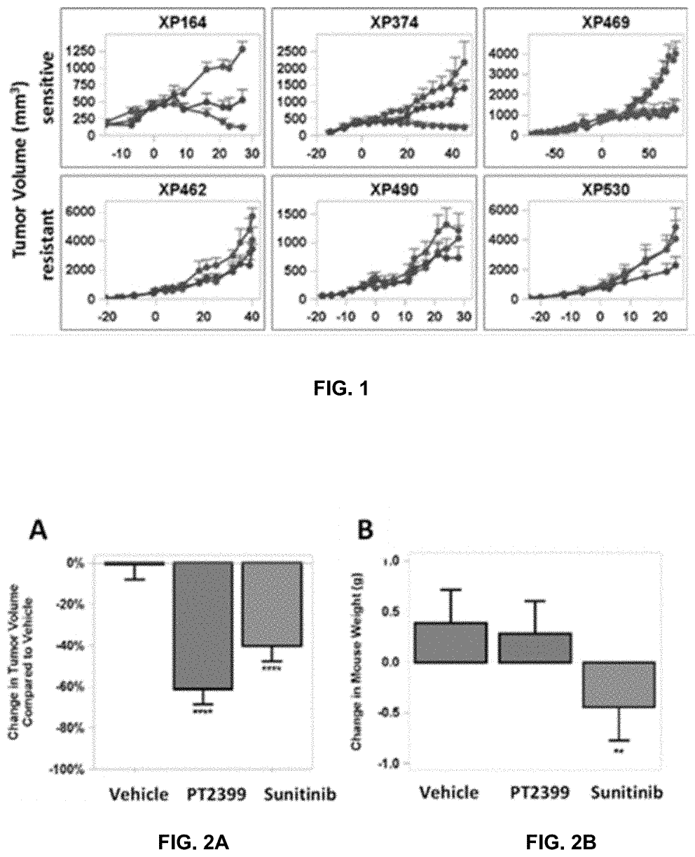

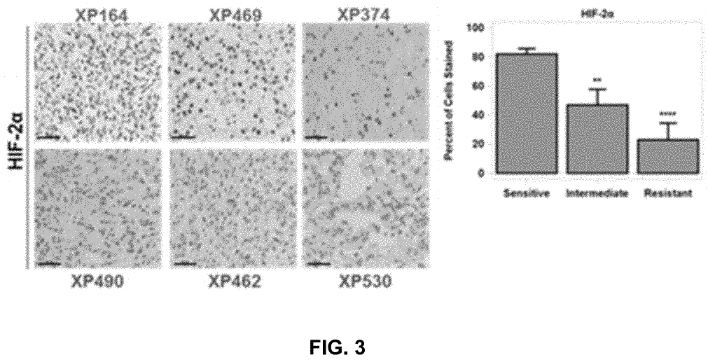

To evaluate the role of HIF-2 inhibition in RCC, HIF-2α inhibitor PT2399 was tested in an extensive tumorgraft (TG or patient-derived xenograft, PDX) platform. This compound was evaluated in over 250 mice implanted with RCCs from 22 patients. As shown in FIG. 1, it was found that approximately 50% of the ccRCC TGs were responsive to PT2399. Notably, PT2399 was found more active than Sunitinib (the standard of care at the time). PT2399 was also better tolerated; as shown in FIG. 2, whereas mice treated with sunitinib—which was administered at doses reproducing patient exposures—lost weight, mice treated with PT2399 did not. This was also observed clinically. Moreover, responsiveness to PT2399 was found to be correlated with HIF-2α expression in tumors (FIG. 3).

### Example 2. Specificity and efficacy of PT2399 as an HIF-2α Inhibitor

As a custom-fit drug lodging into a most unusual internal cavity—internal cavities within proteins are rare as they destabilize tertiary structure—HIF-2α inhibitors were expected to be highly specific. Indeed, it was shown that their effect was specific for HIF-2α and that they did not for example affect the highly related paralogue HIF-1α.

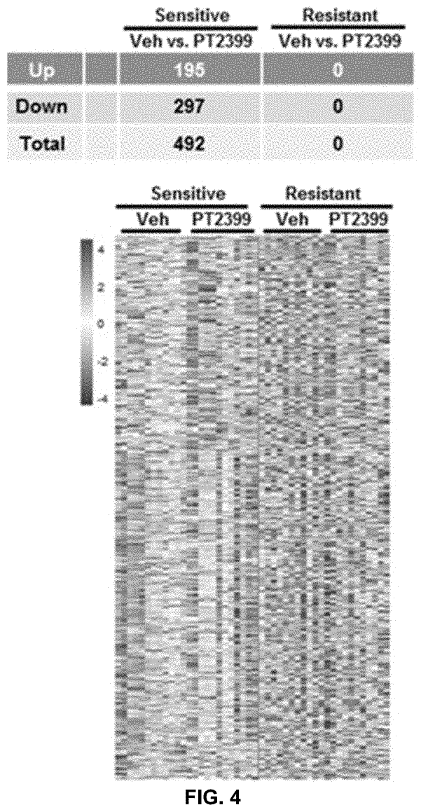

To assess the specificity of PT2399, the impact of the drug on gene expression was measured by RNA-seq. RNA-seq captures the expression of thousands of genes and is a highly sensitive test. It was hypothesized that if the drug were specific, it should not affect tumors devoid of HIF-2α protein. It was found that PT2399, while having profound effects on gene expression in sensitive tumors (HIF-2α expressing tumors), had no effect on resistant tumors lacking HIF-2α expression (FIG. 4). Indeed, while 492 genes were identified as dysregulated in sensitive tumors, no genes were affected by the drug in the resistant tumors. These results were independently reproduced by a third party (The New York Genome Center), who arrived at the same conclusions.

Responsiveness to PT2399 correlated with HIF-2α expression in tumors (FIG. 3), but more importantly, it was found that responsive tumors were characterized by higher HIF-2α protein levels and also a unique gene expression signature enriched for HIF-2α target genes. As expected, ccRCCs that did not express HIF-2α were resistant to PT2399 (FIG. 3).

### Example 3. Evaluation of HIF-2α Inhibitor Efficacy in Human

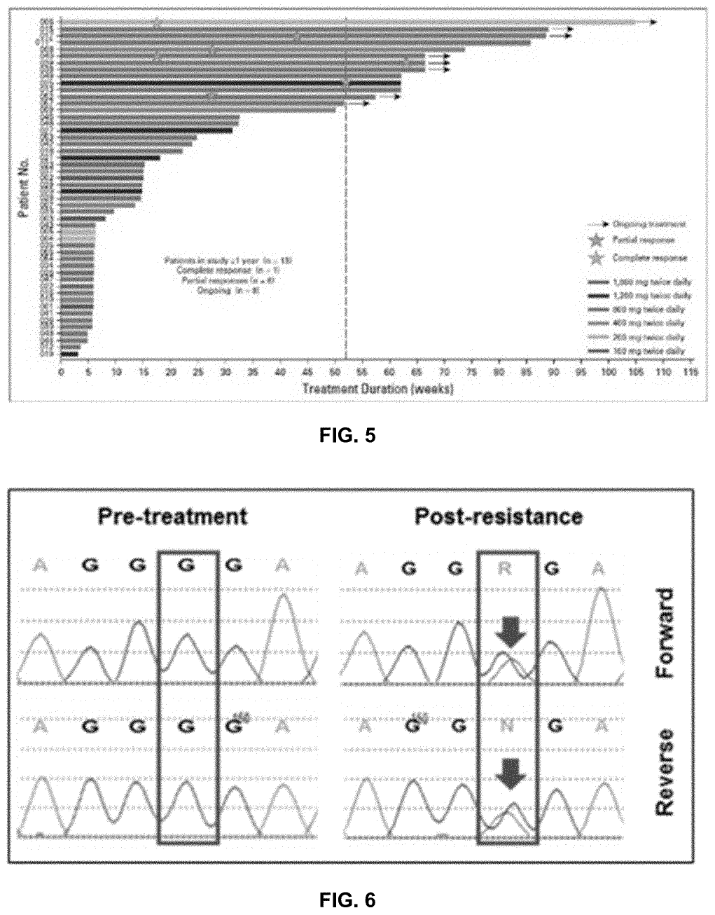

PT2385 (closely related to PT2399) was evaluated in a Phase 1 clinical trial in patients with ccRCC in both a dose escalation and a dose expansion cohort. The drug was remarkably well tolerated and a maximal tolerated dose (MTD) was not reached. In addition, there were no serious adverse effects requiring treatment discontinuation in any patient. The drug was also highly specific, and high-grade adverse effects were on-target, and expected, such as anemia (HIF-2 is known to regulate erythropoietin, and the effect could be overcome by exogenous erythropoietin administration). While the patient cohort evaluated had been extensively pretreated (typically ≥4 prior lines), activity of the inhibitor was still observed. Among 51 patients treated in the study at various dose levels, there was 1 complete response (CR), 6 partial response (PR), and 13 patients who remained on the study without progression beyond a year (FIG. 5). Overall, 42% of patients remained without progression for at least 4 months. Overall, these results were quite similar to those observed previously in tumorgrafts, with respect to both activity and tolerability. Those data illustrated that even in highly pretreated RCC patients the HIF-2 Inhibitor was able to remain active.

In keeping with its specificity, PT2385 was exquisitely well tolerated and there were no dose-limiting toxicities. The Phase I trial was accompanied by a companion study involving imaging and tissue acquisition. As in the tumorgrafts, preliminary studies of tumor samples from patients indicated that activity was related to HIF-2α expression, and there was no activity in HIF-2α deficient tumors.

### Example 4. Evaluation of the Correlation Between Treatment Resistance and Acquisition of HIF-2α Mutation

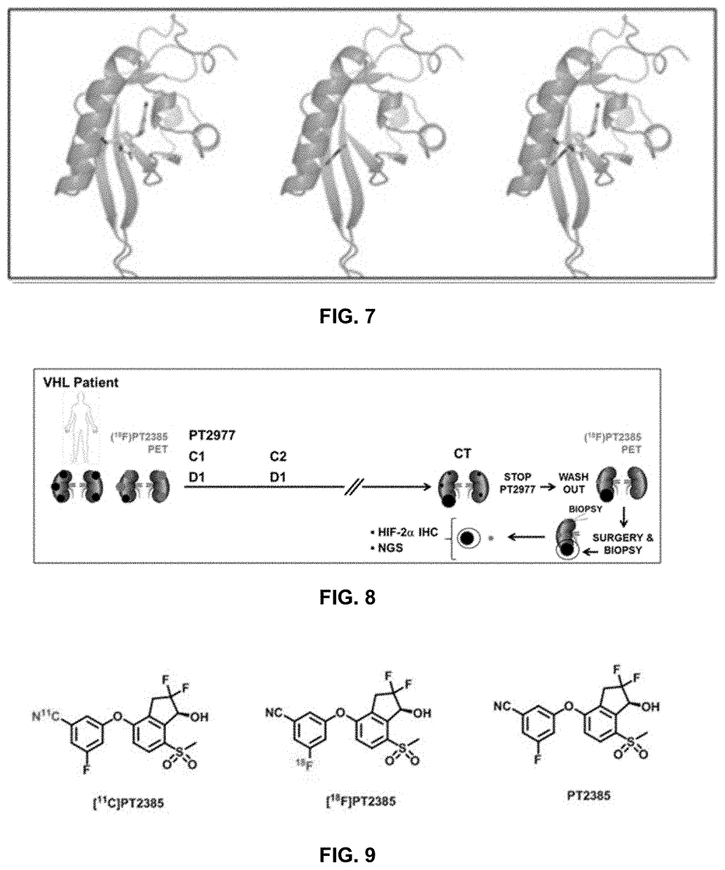

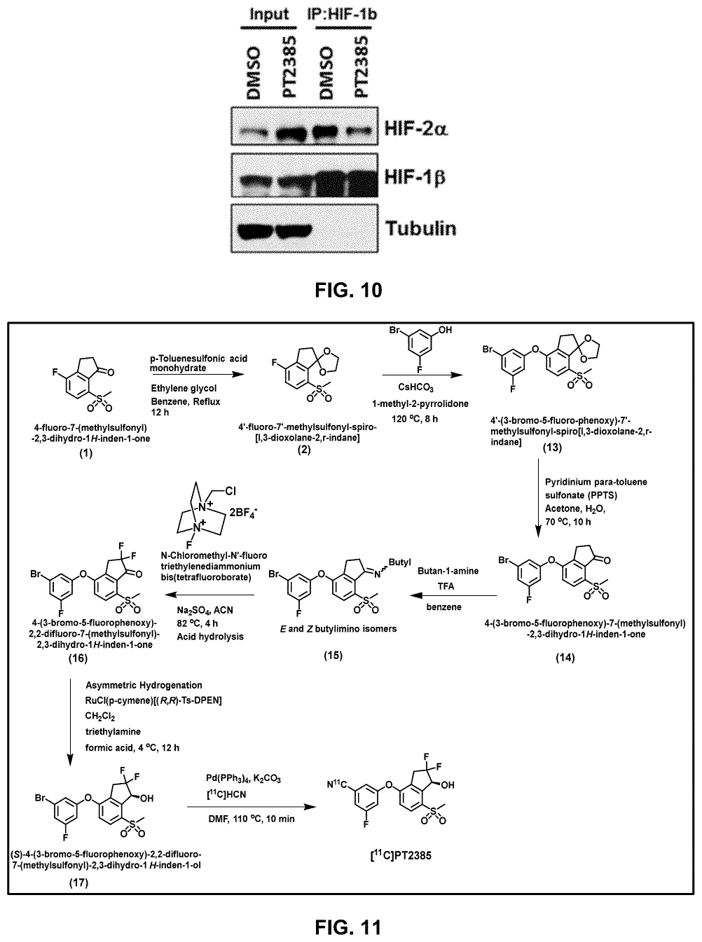

As typically observed with targeted therapies, prolonged treatment of sensitive tumors in tumor-bearing mice led to the acquisition of resistance. Through sequencing analyses, a mutation in a resistant tumor, which was acquired (not present in pre-treatment tumor samples) was identified (FIG. 6). The treatment was applied to sensitive RCC tumorgrafts in mice for over 6 months, which eventually resulted in the development of resistance. Sequencing analyses were performed, and a resistance mutation in the HIF-2α gene was found. The mutation was in the HIF-2α cavity bound by the drug, and it prevented drug binding (FIG. 7). The same mutation was found in a patient who developed resistance after having been on drug for many months. Of note, resistance developed in this patient after one year of therapy, and the patient had been treated with 7 prior lines, showing that PT2385 can have remarkable activity despite the development of resistance. These data added further validation to the on-target specificity of PT2385 and the importance of HIF-2α in ccRCC.

Albeit indirectly, the evaluation of mechanisms of resistance was another way to assess specificity of the targeted therapy: as the PT2385 was developed to target HIF-2α in cells, and as tumors are highly plastic, the administration of the drug applied selective pressure that was ultimately evaded through resistance mutations in the HIF-2α gene, enabling the cells to overcome drug-mediated effects.

### Example 5. Synthesis of [¹¹C]PT2385

After having established that HIF-2α was a valid target in ccRCC, highly specific HIF-2 inhibitors were developed. Inhibitor activity was correlated with HIF-2α expression in tumors and a predictive biomarker to identify patients most likely to benefit from the approach was developed. HIF-2α expression can be measured directly in tissue samples. However, this approach has significant limitations including: (i) relevance—findings in archival samples from tissue obtained sometimes years before the patient developed metastases may not be relevant; (ii) sampling bias—a biopsy may be performed, but this would involve a single site of metastasis, which may not be representative; (iii) heterogeneity—even within a particular site, RCC has been shown to be notoriously heterogeneous and the analyses may not be generalizable.

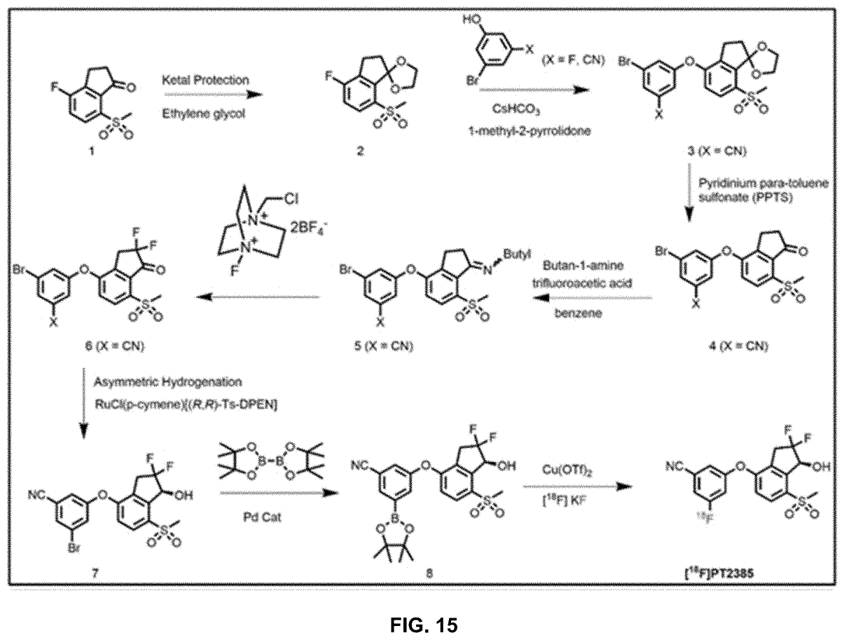

The synthesis of [¹¹C]PT2385 was developed in parallel to the radiosynthesis of [¹⁸F]PT2385. As shown in FIG. 11, the multi-step synthetic route to the precursor (compound 17) for [¹¹C]PT2385 was similar to that for [¹⁸F]PT2385 shown in FIG. 15. Briefly, the synthesis of compound 17 starts from the same starting material 4-fluoro-7-(methylsulfonyl)-2,3-dihydro-1H-inden-1-one (compound 1) and follows the same synthetic route as FIG. 15, in which 3-bromo-5-fluoro-phenol is used instead of 3-fluoro-5-hydroxybenzonitrile. The bromo atom in compound 17 undergoes a [¹¹C]cyanation reaction in the presence of Pd catalyst to produce radiolabeled compound [¹¹C]PT2385.

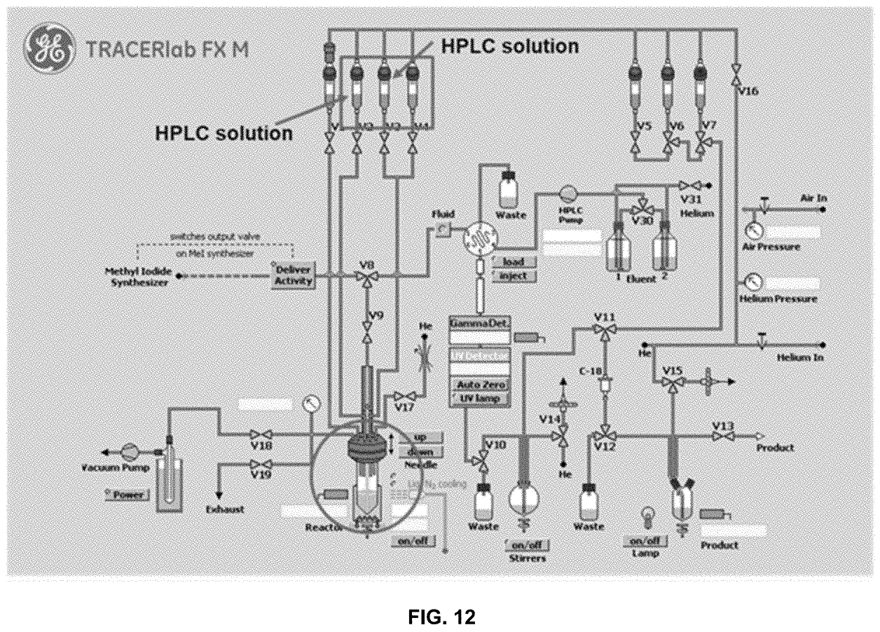

Fully automated procedures were developed for the synthesis of [¹¹C]PT2385. The radiolabeling of compound 17 with ¹¹C by a fully automated procedure in a commercial radiochemistry synthesizer, GE Healthcare's TRACERlab FX M module was accomplished. As shown in FIG. 12, the reactor (purple circle) was equipped with a stir bar and charged with compound 17 (4 mg), [Pd(PPh₃)₄] (5 mg) and K₂CO₃ (1 mg) in 300 pl of DMF. Meanwhile, [¹¹C]HCN was prepared in a GE Healthcare Pro-Cab system and bubbled into the reactor until the radioactivity reached 700-800 mCi. The mixture was heated to 110° C. for 8 min. The reaction mixture was then diluted to 4 mL with HPLC elution medium at room temperature. The entire solution was then transferred to the semi-preparative HPLC for purification, mobile phase (43% CH₃CN/57% H₂O, with 0.1% TFA). The pure fractions containing compound [¹¹C]PT2385 were collected in a flask to mix with 75 mL of water and passed through a C18 Plus Sep-Pak cartridge to trap the radiolabeled compound. The cartridge was then eluted with 5 mL H₂O and then with ethanol (1 mL) to acquire the final product, [¹¹C]PT2385 (˜100 mCi). The total time required to prepare the final dose of [¹¹C]PT2385 was kept under 35 min from the initial time of the radiosynthesis, and >20 trials have already been conducted.

### Example 6. Detection of HIF-2α in RCC Tumorgrafts by PET using [¹¹C]PT2385

Experiments showing that [¹¹C]PT2385 was taken up by ccRCC tumorgrafts expressing HIF-2α and that the uptake was specifically blocked by excess cold PT2385 were performed.

Tumor samples collected fresh from kidney cancer surgeries were implanted orthotopically (in the kidney) of immunocompromised mice (NOD/SCID) within 3 hours. The samples were implanted without disaggregation or any additives, and it was previously shown that these tumorgrafts retained the histology, gene expression, DNA copy number alterations, mutations, and treatment responsiveness of the patient tumors. This platform of human RCC tumorgraft models was used to validate HIF-2α as a target in ccRCC (FIGS. 1 and 2). While tumors were maintained and passaged orthotopically, for terminal drug testing they were implanted subcutaneously (where they can be more easily followed and measured), and it was previously shown that this did not affect their drug responsiveness. This same platform was used to evaluate the uptake of [¹¹C]PT2385 by ccRCC tumorgrafts expressing HIF-2α by PET imaging.

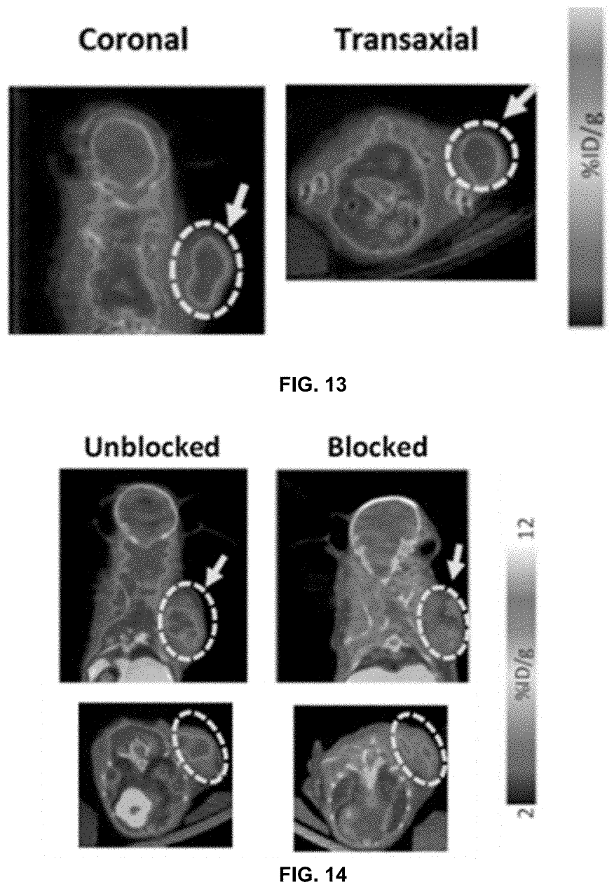

A tumorgraft line expressing high levels of HIF-2α (XP164), which was previously showed as sensitive to HIF-2 inhibition was used as proof-of-principle experiments with [¹¹C]PT2385. Tumor-bearing mice were anesthetized using isofluorane, injected with 100-150 μCi of [¹¹C]PT2385 via the tail vein, and subjected to imaging with a whole-body CT scan (80 keV, 500 pAmp, 140 ms exposure) and static PET scans at different time points. CT images were reconstructed using the Feldkamp algorithm and Shepp-Logan filter with beam-hardening correction. List-mode data were histogrammed and reconstructed into single-frame (128×128 matrix size) PET images using OSEM3D/SP-MAP (2 OSEM iterations, 18 MAP iterations, and 1.5 mm target resolution) reconstruction algorithm. PET and CT images were overlapped and coregistered (Siemens Medical Imaging). Quantitation values were obtained as average % injected dose per gram (% ID/g) from the volume encompassing all slices containing the regions of interest on the fused PET/CT images. As shown in FIG. 13, [¹¹C]PT2385 uptake in ccRCC patient explants expressing high HIF-2α levels in mice was detected.

To determine whether the tumor uptake observed was specific, blocking experiments were performed. For these experiments, [¹¹C]PT2385 was co-injected with 1000-fold excess of cold PT2385. As shown in FIG. 14, the tumor uptake of [¹¹C]PT2385 was significantly reduced by the co-injection of unlabeled PT2385, indicative of the desired HIF-2α imaging specificity of PET with [¹¹C]PT2385. These data provide a proof-of-principle for the use of radiolabled PT2385 as a probe to evaluate HIF-2α protein in tumors.

### Example 7. Design and Synthesis of [¹⁸F]PT2385

The design of [¹⁸F]PT2385 maintained the same chemical structure of the parent compound, PT2385 (FIG. 9), so that (i) the specific binding affinity to HIF-2α was not affected, and (ii) the toxicology and pharmacology data of PT2385 can be referenced for regulatory filings.

As outlined in FIG. 15, the multi-step synthetic route to [¹⁸F]PT2385 started from 4-fluoro-7-(methylsulfonyl)-2,3-dihydro-1H-inden-1-one compound 1, which is commercially available. Briefly, its ketone group was protected in the presence of ethane-1,2-diol to form a cyclic ketal compound 2, which then underwent a nucleophilic aromatic substitution with 3 bromo-5-hydroxybenzonitrile (X=CN) to form compound 3. Deprotection of the cyclic ketal group in compound 3 in the presence of pyridinium p-toluenesulfonate produced compound 4, which after condensation with n-butylamine yielded a mixture butylimino isomers as compound 5, in which the imine bond may present E and Z isomers. Fluorination of compound 5 using N-chloromethyl-N′-fluorotriethylenediammonium bis(tetrafluoroborate) provided compound 6 after acid hydrolysis. Asymmetric hydrogenation of compound 6 produced compound 7. Expected enantiomeric excess was 98%. By a Pd catalytic reaction with bis(pinacolato)diboron, compound 7 was converted to compound 8, the desired precursor to make (¹⁸F)PT2385 via reacting with [¹⁸F]KF in presence of Cu(OTf)₂(Py)₂.

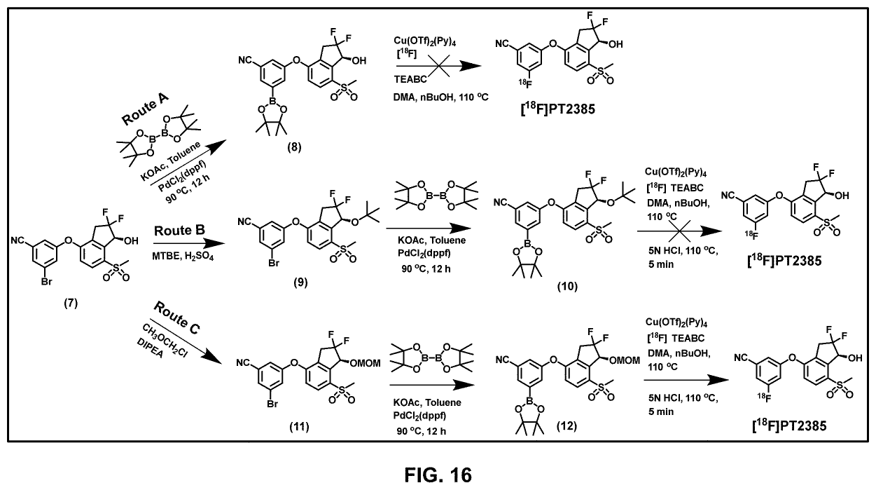

However, a trace amount of borates remaining in compound 8 made the purification from the reaction media challenging. Alternative routes (route B; and Route C FIG. 16) were thus investigated. Route B led to the production of ^{t}Bu-protected [¹⁸F]PT2385 in yields up to 10% (decay-corrected) and [¹⁸F]PT2385 after 20 min of deprotection of ^{t}Bu group. While this was successful, to improve the yield, another alternative route was developed (route C; FIG. 16). Incorporation of methoxymethyl ether (MOM) protecting group produces compound (11). Compound (11) was then reacted with bis(pinacolato)diboron in presence of PdC1₂(dppf) catalyst to produce the precursor compound (12), and purified via silica gel column chromatography. Radiolabeling of compound (12) yielded MOM-protected [¹⁸F]PT2385, which after acid treatment at 110° C. for 5 minutes resulted in the HIF-2α radiotracer [¹⁸F]PT2385.

### Example 8. Automated Radiosynthesis of [¹⁸F]PT2385

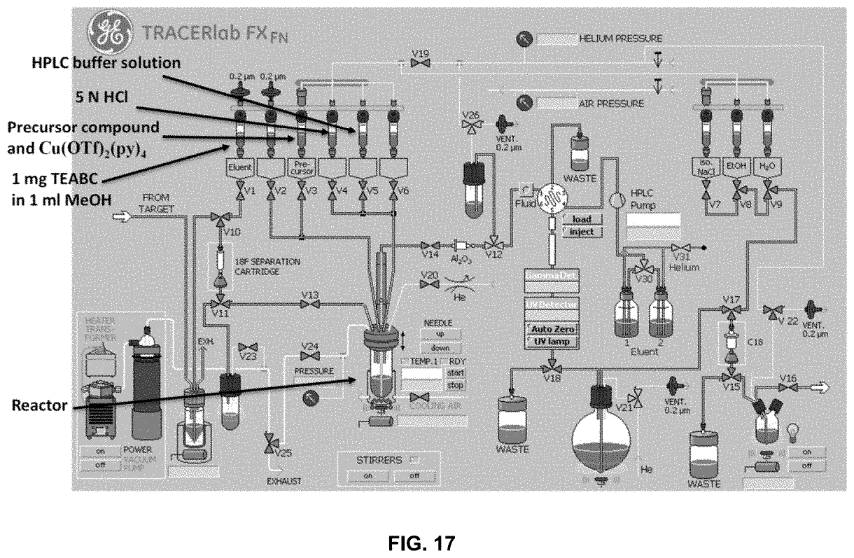

Similar to the automated radiosynthesis of [¹¹C]PT2385, the automated radiosynthesis of [¹⁸F]PT2385 has been developed in a commercial nucleophilic radiofluorination synthesizer, a GE Healthcare FX N Pro Module (FIG. 17). Typically, up to 1.5 Ci of [¹⁸F]fluoride in 2 mL of target water is passed through a QMA Light Sep-Pak cartridge to trap the entire activity. [¹⁸F]Fluoride is then eluted out from the cartridge into Reactor 1 of TRACERlab FX N pro module with the eluent (1.0 mg Et₄NHCO₃ in 1.0 mL methanol) in vial 1, and azeotropically dried at 50° C. for 2 min, then at 110° C. for 10 min under N₂/vacuum. After cooling the reaction vessel down to 50° C., a solution of the chosen precursor, compound 10 or 12 (5.0 mg) and Cu(OTf)₂(py)₄ (15.0 mg) in 0.5 mL nBuOH/DMA 1/2 in Vial 3 is added, and the reaction mixture is then heated for 10 min at 110° C. After cooling the reaction vessel to 30° C., the reaction mixture will be analyzed for the product formation in terms of radiochemical yield and purity as well as radionuclidic identity/purity.

### Example 9. Detection of HIF-2α in RCC Tumorgrafts by PET using [¹⁸F]PT2385

Similar to [¹¹C]PT2385, [¹⁸F]PT2385 was tested in tumorgraft mouse models generated from patients with kidney cancer to establish and validate the proposed imaging methods.

Tumorgraft models with high and low HIF-2α levels were administered [¹⁸F]PT2385 and imaged with PET/CT. Tumors were excised for post-imaging gamma counting and sectioned for immunochemical (IHC) staining with anti-HIF-2α to validate the correlation between imaging signal readouts and expression of HIF-2α protein. Radiation dosimetry was assessed by analyzing the bio-distribution data obtained from PET imaging along with the clearance data.

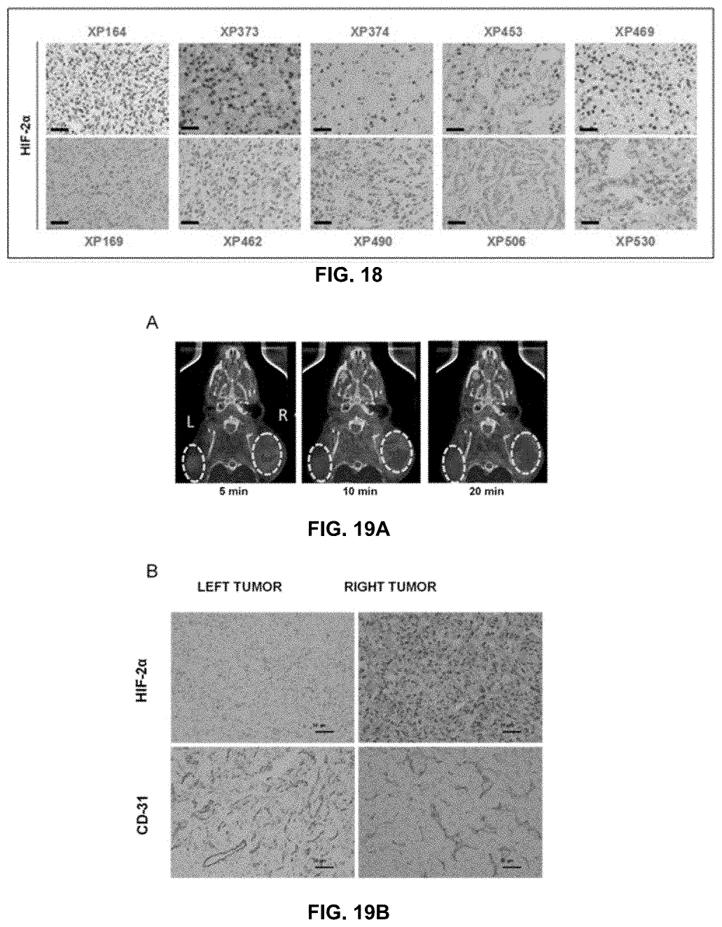

As shown in FIG. 18, a large number of tumorgraft lines with both high and absent/low levels of HIF-2α have already been collected. A subset of these lines, in which HIF-2α expression largely correlates with sensitivity to HIF-2α inhibitors (Table 1) can be used for the detection of HIF-2α in RCC tumorgrafts by PET using [¹⁸F]PT2385.

TABLE 1
List of HIF-2α inhibitor sensitive and resistant RCC
tumorgraft lines with corresponding HIF-2α expression levels.

| Response | XP NO. | HIF-2α IHC |
|---|---|---|
| Sensitive | XP26 | 80 |
| | XP144 | 80 |
| | XP164 | 80 |
| | XP165 | 70 |
| | XP373 | 100 |
| | XP374 | 95 |
| | XP453 | 100 |
| | XP454 | 60 |
| | XP469 | 90 |
| | XP534 | 80 |
| Resistant | XP169 | 10 |
| | XP462 | 0 |
| | XP490 | 10 |
| | XP506 | 0 |
| | XP530 | 10 |

As shown in FIG. 19, [¹⁸F]PT2385 is specific for the detection of HIF-2α expressing tumors. For these experiments, mice were implanted with two tumors (ccRCC) with different levels of HIF-2α. On the left shoulder, tumors were implanted devoid of HIF-2α expression (XP534) (L). On the right shoulder, tumors were implanted that expressed HIF-2α (XP164) (R). Subsequently, mice were injected with [¹⁸F]PT2385 by tail vein and PET images were acquired at different times. As shown in FIG. 19A, the high HIF-2α expressing tumors were recognized by the [¹⁸F]PT2385 tracer, but not the low expressing tumors implanted on the left side. The differential HIF-2α expression levels between the two tumor lines were confirmed by immunohistochemistry (FIG. 19B). Importantly, as shown by a CD-31 immunohistochemistry, both tumor lines had similar vascularity (based on CD-31 staining). Overall, these data show that [¹⁸F]PT2385 PET is able to distinguish tumors with differential expression of HIF-2α.

### REFERENCES

1. Erbel, P. J., P. B. Card, O. Karakuzu, R. K. Bruick, and K. H. Gardner, Structural basis for PAS domain heterodimerization in the basic helix—loop—helix-PAS transcription factor hypoxia-inducible factor. Proc Natl Acad Sci USA, 2003. 100(26): p. 15504-9.

2. Scheuermann, T. H., D. R. Tomchick, M. Machius, Y. Guo, R. K. Bruick, and K. H. Gardner, Artificial ligand binding within the HIF2alpha PAS-B domain of the HIF2 transcription factor. Proc Natl Acad Sci USA, 2009. 106(2): p. 450-5.

3. Tian, H., R. E. Hammer, A. M. Matsumoto, D. W. Russell, and S. L. McKnight, The hypoxia-responsive transcription factor EPAS1 is essential for catecholamine homeostasis and protection against heart failure during embryonic development. Genes Dev, 1998. 12(21): p. 3320-4.

4. Chen, W., H. M. Hill, A. Christie, M. S. Kim, E. Holloman, A. Pavia-Jimenez, F. Homayoun, Y. Ma, N. Patel, P. Yell, G. Hao, Q. Yousuf, K. Gardner, R. Bruick, K. Courtney, E. Frenkel, I. Pedrosa, X. Sun, T. H. Hwang, T. Wong, J.P. Rizzi, E. M. Wallace, J. J. A., X. Y., X. J. Xie, P. Kapur, R. M. McKay, and J. Brugarolas, Targeting Renal Cell Carcinoma with an HIF-2 antagonist. Nature, 2016. 539(7627): p. 112-17.

5. Courtney, K. D., J. R. Infante, E. T. Lam, R. A. Figlin, B. I. Rini, J. Brugarolas, N. J. Zojwalla, A. M. Lowe, K. Wang, E. M. Wallace, J. A. Josey, and T. K. Choueiri, Phase I Dose-Escalation Trial of PT2385, a First-in-Class Hypoxia-Inducible Factor-2alpha Antagonist in Patients With Previously Treated Advanced Clear Cell Renal Cell Carcinoma. J Clin Oncol, 2017: p. JCO2017742627.

6. Scheuermann, T. H., Q. Li, H. W. Ma, J. Key, L. Zhang, R. Chen, J. A. Garcia, J. Naidoo, J. Longgood, D. E. Frantz, U. K. Tambar, K. H. Gardner, and R. K. Bruick, Allosteric inhibition of hypoxia inducible factor-2 with small molecules. Nat Chem Biol, 2013. 9(4): p. 271-6.

7. Tian, H., S. L. McKnight, and D. W. Russell, Endothelial PAS domain protein 1 (EPAS1), a transcription factor selectively expressed in endothelial cells. Genes Dev, 1997. 11(1): p. 72-82.

8. Kondo, K., W. Y. Kim, M. Lechpammer, and W. G. Kaelin, Jr., Inhibition of HIF2alpha is sufficient to suppress pVHL-defective tumor growth. PLoS Biol, 2003. 1(3): p. E83.

9. Shen, C. and W. G. Kaelin, Jr., The VHL/HIF axis in clear cell renal carcinoma. Semin Cancer Biol, 2013. 23(1): p. 18-25.

10. Gerlinger, M., A. J. Rowan, S. Horswell, J. Larkin, D. Endesfelder, E. Gronroos, P. Martinez, N. Matthews, A. Stewart, P. Tarpey, I. Varela, B. Phillimore, S. Begum, N. Q. McDonald, A. Butler, D. Jones, K. Raine, C. Latimer, C. R. Santos, M. Nohadani, A. C. Eklund, B. Spencer-Dene, G. Clark, L. Pickering, G. Stamp, M. Gore, Z. Szallasi, J. Downward, P. A. Futreal, and C. Swanton, Intratumor heterogeneity and branched evolution revealed by multiregion sequencing. N Engl J Med, 2012. 366(10): p. 883-92.

11. Bensch, F., E. L. van der Veen, M. N. Lub-de Hooge, A. Jorritsma-Smit, R. Boellaard, I. C. Kok, S. F. Oosting, C. P. Schroder, T. J. N. Hiltermann, A. J. van der Wekken, H. J. M. Groen, T. C. Kwee, S. G. Elias, J. A. Gietema, S. S. Bohorquez, A. de Crespigny, S. P. Williams, C. Mancao, A. H. Brouwers, B. M. Fine, and E. G. E. de Vries, (89)Zr-atezolizumab imaging as a non-invasive approach to assess clinical response to PD-L1 blockade in cancer. Nat Med, 2018.

12. Joshi-Pangu, A., X. Ma, M. Diane, S. Iqbal, R. J. Kribs, R. Huang, C. Y. Wang, and M. R. Biscoe, Palladium-catalyzed borylation of primary alkyl bromides. J Org Chem, 2012. 77(15): p. 6629-33.

13. van der Born, D., A. Pees, A. J. Poot, R. V. A. Orru, A. D. Windhorst, and D. J. Vugts, Fluorine-18 labelled building blocks for PET tracer synthesis. Chem Soc Rev, 2017. 46(15): p. 4709-4773.

What is claimed is:

1.-33. (Canceled)

34. A method of detecting pulmonary hypertension in a subject comprising:
a) administering to the subject a hypoxia inducible factor 2-alpha (HIF-2α)-specific radioactive tracer comprising an HIF-2α-specific inhibitor and a radioactive label, wherein the radioactive label is a positron emitting radioisotope;
b) subjecting the subject to a positron emission topography (PET) scan; and
c) determining an amount of the tracer, wherein an increased amount of the tracer as compared to a control indicates that the subject has pulmonary hypertension.

35. The method of claim 34, further comprising administering to the subject having pulmonary hypertension a pulmonary hypertension treatment.

36. A method of monitoring pulmonary hypertension in a subject comprising:
a) administering to a subject having pulmonary hypertension a hypoxia inducible factor 2-alpha (HIF-2α)-specific radioactive tracer comprising an HIF-2α-specific inhibitor and a radioactive label, wherein the radioactive label is a positron emitting radioisotope, subjecting the subject to a first positron emission topography (PET) scan, and determining a first amount of HIF-2α expression in the subject;
b) at a period of time after step a) administering to the subject the HIF-2α-specific radioactive tracer of claim 1, subjecting the subject to a second PET scan, and determining a second amount of HIF-2α expression in the subject; and
d) comparing the first amount and second amount of HIF-2α expression, thereby monitoring pulmonary hypertension evolution in the subject over time.

37. The method of claim 36, wherein where the second amount of HIF-2α expression is increased as compared to the first amount of HIF-2α then there is no improvement of the pulmonary hypertension in the subject, wherein where the second amount of HIF-2α expression is the same as compared to the first amount of HIF-2α expression, then there is a stabilization of the pulmonary hypertension, and wherein where the second amount of HIF-2α expression is decreased as compared to the first amount of HIF-2α then there is improvement in the pulmonary hypertension.

38. A method of evaluating efficacy of a pulmonary hypertension treatment in a subject comprising:
a) administering to a subject having pulmonary hypertension a hypoxia inducible factor 2-alpha (HIF-2α)-specific radioactive tracer comprising an HIF-2α-specific inhibitor and a radioactive label, wherein the radioactive label is a positron emitting radioisotope, subjecting the subject to a first positron emission topography (PET) scan, and determining a first amount of HIF-2α expression in the subject;
b) administering to the subject a pulmonary hypertension treatment;
c) administering to the subject the HIF-2α-specific radioactive tracer of claim 1, subjecting the subject to a second PET scan, and determining a second amount of HIF-2α expression in the subject; and
d) comparing the first amount and second amount of HIF-2α expression, wherein where a second amount of the HIF-2α-specific radioactive tracer is decreased as compared to the first amount, then there is efficacy of the pulmonary hypertension treatment.

39. The method of claim 38, wherein the pulmonary hypertension treatment comprises an HIF-2α inhibitor.

40. The method of claim 38, wherein a second amount of the HIF-2α-specific radioactive tracer is equivalent or increased as compared to the first baseline level indicates that there is no efficacy of the pulmonary hypertension treatment.

41. The method of claim 34, wherein the pulmonary hypertension is a HIF-2α-mediated pulmonary hypertension.

42. The method of claim 34, wherein administering the HIF-2α-specific radioactive tracer is by intravenous, intraarterial, or oral administration.

43. The HIF-2α-specific radioactive tracer of claim 34, wherein the positron emitting radioisotope is ¹¹C or ¹⁸F.

44. The HIF-2α-specific radioactive tracer of claim 34, wherein the HIF-2α-specific inhibitor is

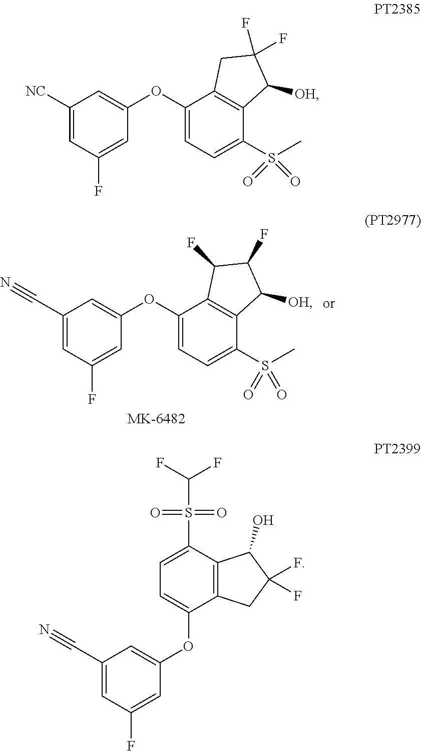 [SMILES: CS(=O)(=O)c1ccc(Oc2cc(F)cc(C#N)c2)c2c1[C@H](O)C(F)(F)C2 ; CS(=O)(=O)c1ccc(Oc2cc(F)cc(C#N)c2)c2c1[C@H](O)[C@H](F)[C@@H]2F ; N#Cc1cc(F)cc(Oc2ccc(S(=O)(=O)C(F)F)c3c2CC(F)(F)[C@H]3O)c1]

45. The HIF-2α-specific radioactive tracer of claim 34, wherein the HIF-2α-specific radioactive tracer is

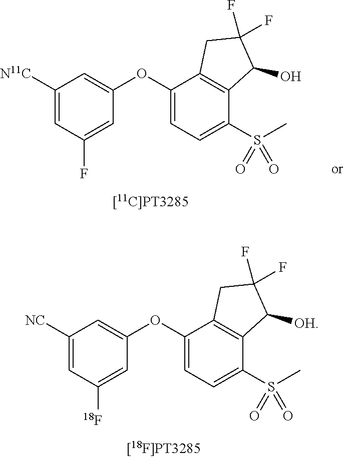

46. The HIF-2α-specific radioactive tracer of claim 36, wherein the positron emitting radioisotope is ¹¹C or ¹⁸F.

47. The HIF-2α-specific radioactive tracer of claim 36, wherein the HIF-2α-specific inhibitor is

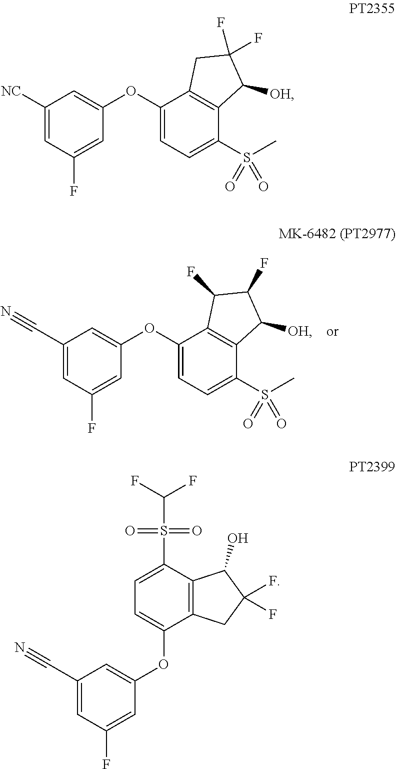 [SMILES: CS(=O)(=O)c1ccc(Oc2cc(F)cc(C#N)c2)c2c1[C@H](O)C(F)(F)C2 ; CS(=O)(=O)c1ccc(Oc2cc(F)cc(C#N)c2)c2c1[C@H](O)[C@H](F)[C@@H]2F ; N#Cc1cc(F)cc(Oc2ccc(S(=O)(=O)C(F)F)c3c2CC(F)(F)[C@H]3O)c1]

48. The HIF-2α-specific radioactive tracer of claim 36, wherein the HIF-2α-specific radioactive tracer is

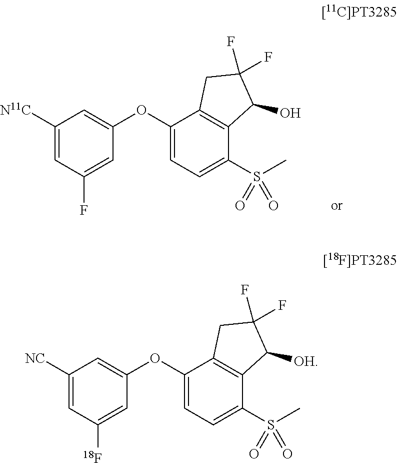

49. The HIF-2α-specific radioactive tracer of claim 38, wherein the positron emitting radioisotope is ¹¹C or ¹⁸F.

50. The HIF-2α-specific radioactive tracer of claim 38, wherein the HIF-2α-specific inhibitor is

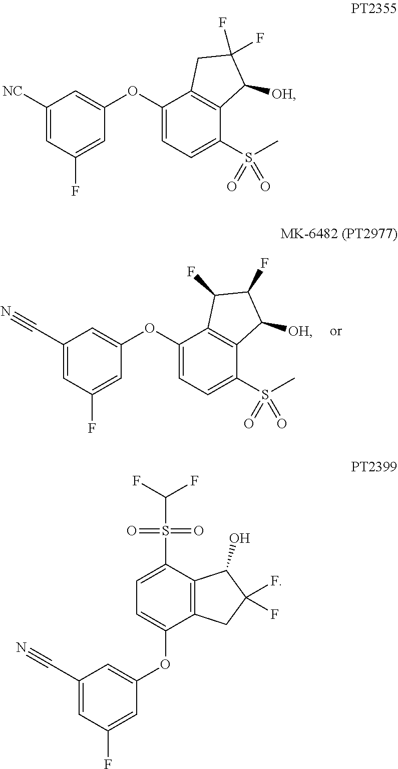 [SMILES: CS(=O)(=O)c1ccc(Oc2cc(F)cc(C#N)c2)c2c1[C@H](O)C(F)(F)C2 ; CS(=O)(=O)c1ccc(Oc2cc(F)cc(C#N)c2)c2c1[C@H](O)[C@H](F)[C@@H]2F ; N#Cc1cc(F)cc(Oc2ccc(S(=O)(=O)C(F)F)c3c2CC(F)(F)[C@H]3O)c1]

51. The HIF-2α-specific radioactive tracer of claim 38, wherein the HIF-2α-specific radioactive tracer is

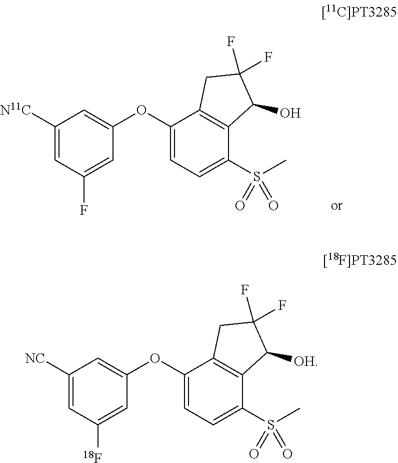

52. The method of claim 36, wherein administering the HIF-2α-specific radioactive tracer is by intravenous, intraarterial, or oral administration.

53. The method of claim 38, wherein administering the HIF-2α-specific radioactive tracer is by intravenous, intraarterial, or oral administration.
